(19) United States
(12) Patent Application Publication
(10) Pub. No.: US 2026/0240683 A1
(43) Pub. Date: Aug. 20, 2026

(54) MOLECULAR LINKERS

(71) Applicant: CWAN TECHNOLOGIES PTY LTD, Tennyson (AU)

(72) Inventors: Nancy Liyou, Tennyson (AU); Christopher Elvin, Tennyson (AU)

(21) Appl. No.: 19/161,733

(22) PCT Filed: Mar. 2, 2024
(86) PCT No.: PCT/AU2024/050174

§ 371 Date: Sep. 3, 2025

(30) Foreign Application Priority Data
Mar. 3, 2023 (AU) 2023900571

(51) Int. Cl. A61F 9/007; A61F 9/00; A61K 41/00; A61P 27/02

(52) U.S. Cl. CPC A61F 9/0079; A61F 9/0017; A61K 41/0057; A61P 27/02

(57) ABSTRACT

The present disclosure relates to the crosslinking of endogenous proteins in a tissue using molecular linkers, and therapeutic compositions comprising the linkers. The present disclosure further relates to therapeutic methods and therapeutic compositions for the treatment of diseases and disorders.

### TECHNICAL FIELD

The present disclosure relates to the crosslinking of endogenous proteins in a tissue using molecular linkers, and therapeutic compositions comprising molecular linkers. The present disclosure further relates to therapeutic methods and therapeutic compositions for the treatment of diseases and disorders.

### BACKGROUND

Ectatic corneal disease (ECD) or corneal ectasia is a group of progressive eye disorders characterized by progressive corneal steepening and thinning. Corneal ectasias are associated with decreased uncorrected visual acuity (UCVA), an increase in ocular aberrations, and often a loss of best-corrected distance visual acuity. Corneal ectasias can result in significant ocular morbidity and may require surgical intervention.

Corneal ectasias like Keratoconus (KC), pellucid marginal degeneration, and post-refractory or posttraumatic corneal ectasia are characterised by progressive corneal steepening, thinning and refractive changes. Keratoconus is a bilateral, progressive, degenerative corneal condition resulting in a degenerative thinning and weakening of the corneal extracellular matrix (ECM), with consequent bulging of the cornea and progressive loss of vision. It is characterized by progressive, noninflammatory, inferior corneal thinning, steepening, and apical scarring. Affecting around 2-3 per 1000 people in the developed world, it is the second most common reason for corneal transplants globally. Corneal ectasias can severely impede an individual's sight and quality of life and are potentially disabling.

While the etiology of keratoconus is not fully understood, interactions of genetic factors with environmental stressors are suspected. Without treatment, approximately 20% of patients require corneal transplantation, with some countries reporting keratoconus as the leading indication, accounting for 40% to 45% of corneal transplants annually. Several treatment options are available to patients with KC. These include refractive correction (with glasses or contact lenses), implantation of intracorneal ring segments, and, in advanced stages, corneal transplantation (penetrating keratoplasty or deep anterior lamellar keratoplasty). Although such approaches can be effective, they can be costly, and dependent upon skilled surgeons for good outcomes. Furthermore, corneal grafting requires patients to take long-term immunosuppressant drugs, and the procedure may place patients at an increased life-time risk of developing globe rupture due to its weakened architecture.

Corneal crosslinking (CXL) represents a relatively recent method that can avoid the more drastic surgical step of corneal transplantation. It has been approved by the FDA for the treatment of KC and corneal ectasia. The Dresden protocol, which was the CXL technique initially described for treatment of Keratoconus, involves 30 min of ultraviolet-A (UVA at 370 nm) irradiation at an irradiance of 3.0 mW/cm², resulting in a total surface dose of 5.4 J/cm² (Wollensak et al., 2003). The common understanding is that exposure of Riboflavin (Rf) to UV-A light, in an oxygenated aqueous environment, causes the formation of the highly reactive singlet oxygen radical, which then interacts with corneal tissue protein components to produce additional cross-linked bonds (Kamaev et al., 2012). Based on the Bunsen-Roscoe law of reciprocity, the same photochemical effect can be achieved with a reduced illumination time and a correspondingly increased irradiation intensity. Several new commercially available CXL devices offer higher UVA irradiances, which allow shorter treatment times with delivery of similar energy (Dervenis et al., 2020). For example, an accelerated crosslinking (A-CXL) protocol now available involves 18 min of pulsed UVA (370 nm) in (20 mW/cm², 7.2 J/cm², pulsed profile: 1 sec on, 2 sec off). An alternative A-CXL protocol involves a 9 mW/cm² UV-A irradiation for 10 minutes, without light pulsing. The standard CXL protocol (Wollensak et al., 2003; Spoerl et al., 1998) consists of riboflavin (vitamin B2) applied to a de-epithelized cornea for 30 min. to facilitate molecular diffusion into the stroma to a depth of ~200 μm. Diffusion of riboflavin plays a key role in the CXL procedure, with axial corneal diffusion for common epi-off CXL has been found to equal 6.5×10⁻⁵ mm²/sec, with a typical diffusion time of 30 min (Seiler et al., 2014).

The rationale behind this approach is that UV-A induced photo-polymerization, in the presence of the photosensitizer riboflavin, leads to creation of covalent bonds between substrates within the corneal stroma, including between collagens and proteoglycans and other stromal proteins (Wollensak et al., 2003; Brummer et al., 2011; Hayes et al., 2013). The effects of these induced cross-links have been variously reported as increases in tissue stiffness, resistance to enzymatic digestion, changes to ultrastructure, and altered swelling behaviour (Akhtar et al., 2013; Spoerl et al., 2004a; Spoerl et al., 2004b; Wollensak et al., 2004). Mechanical properties of biological tissues are largely dependent on the intertwining of collagen fibrils, linked lamina layers, and interfibrillar spacing. Corneal collagen crosslinking (CXL) can provide stiffening of the cornea and possibly alter the natural history of KC. It is a well-established method and can reduce the risk of KC progression in the long term (Raiskup-Wolf et al., 2008; O'Brart et al., 2015).

The CXL method, however, operates only at short range (<5 Å) molecular distances, since this crosslinking protocol relies on local diffusion of highly reactive singlet oxygen species formed by the UV-illumination of riboflavin. Hayes et al., (2013) concluded that riboflavin/UVA therapy does not result in the wide-spread cross-linking of collagen molecules. Further, evidence was provided that riboflavin/UVA-induced cross-links do not exist between or within collagen fibrils, but possibly occur at the surface of individual collagen molecules comprising fibrils and within proteoglycan rich coating surrounding the collagen fibrils.

Despite its FDA approval and widespread clinical acceptance for KC treatment, the current riboflavin/UV-A CXL approach suffers from several real and potential drawbacks.

(i) The use of potentially DNA-damaging UV irradiation (illumination at a wavelength of 370 nm) with adverse and harmful effects on endothelium cells.

(ii) Production of highly reactive and non-specific singlet oxygen free radicals. These free radicals can cause damage to sensitive endothelial cells and keratocytes. Potential damage to endothelial cells is avoided by preventing extensive diffusion of riboflavin to the deeper (>300 μm) layers of the corneal stroma and restricting the total dose of UV energy to a biologically safe level (<5.4 J/cm²). This method is dependent on the diffusion and stromal availability of high concentrations of molecular oxygen, the substrate for the photochemical production of reactive singlet oxygen species. Since the Dresden photochemical crosslinking technique consumes molecular oxygen, the treatment process is necessarily time-consuming (requiring approximately 9 to 18 minutes of irradiation per eye treatment), even with the use of various pulsed laser methods allowing time during the irradiation process for maintenance of sufficiently high levels of oxygen within the stromal matrix.

(iii) Complications of corneal cross-linking include corneal haze, corneal scarring, infective keratitis, sterile infiltrates, delayed epithelial healing, failure of treatment, excessive corneal flattening with hyperopic shift and endothelial failure.

(iv) Corneal endothelial damage may occur if the safety limits regarding corneal thickness to prevent endothelial toxicity are not adhered to. Sharma et al. (2012) reported a 1.4% incidence of persistent endothelial failure in 350 eyes treated with the standard epithelium-off protocol although the safety limit of corneal thicknesses of greater than 400 um (epithelium-off) were adhered to (Vimalin et al., 2012). This could be due to intraoperative stromal dehydration resulting in stromal thinning, lack of homogeneity and focusing/alignment issues of the UV devices.

Contraindications and complications for CXL procedures have been described, including: corneal infection; corneal sensitivity and tear function; effect on limbal epithelial cells; transient and persistent corneal stromal haze and sterile infiltrates; endothelial toxicity and thin corneas; corneal drug penetrance and IOP measurements; changes in corneal thickness; post-operative pain; infectious keratitis; development of a persistent epithelial defect; development of a persistent epithelial defect; stromal scarring, corneal melt; Corneal endothelial decompensation and damage to the lens or retina; and development of late onset peripheral ulcerative keratitis.

Accordingly, there remains a need for methods of crosslinking endogenous proteins in tissues, and methods of treating or preventing ectatic corneal diseases such as keratoconus, and/or for strengthening the cornea.

### SUMMARY

The present disclosure is based on the inventors' identification of molecular linkers suitable for crosslinking endogenous proteins in a tissue. The present inventors have demonstrated that the inclusion of a molecular linker polymer, also referred to herein as a ‘molecular linker’ or ‘molecular staple’, with a suitable molecular weight and containing two or more crosslinking functional groups, for example such as tyramine or tyrosine, facilitates protein crosslinking of more distantly located endogenous protein molecules in a tissue. The molecular linkers, and crosslinking method described herein, have use in the treatment of disease.

Accordingly, in one aspect, there is provided a method of crosslinking endogenous protein in a tissue, the method comprising:
(i) contacting the tissue with a molecular linker, a photoactivatable metal ligand complex, and an electron acceptor, whereby the molecular linker contacts the endogenous protein in the tissue; and
(ii) irradiating the tissue.

Irradiating the tissue in the presence of the molecular linker, photoactivatable metal ligand complex and electron acceptor results in the crosslinking of the molecular linker and the endogenous protein.

In certain embodiments, the molecular linker is a polymer comprising two or more tyrosine and/or tyramine residues.

In other embodiments, the polymer comprising one or more tyrosine and/or tyramine residues may be selected from tyrosine and/or tyramine-derivatised gelatin, tyrosine and/or tyramine-derivatised dextran, and/or tyrosine and/or tyramine-derivatised xylan and/or tytosine and/or tyramine-derivatised low molecular weight nanocellulose.

In some embodiments, the molecular linker has a molecular weight between about 5 kDa to about 50 kDa, or about 5 kDa to 40 kDa. Alternatively, the molecular linker has a molecular weight between about 10 kDa to about 20 kDa, or about 12 kDa to about 20 kDa, or about 10 kDa to about 15 kDa.

In some embodiments of the crosslinking method, the photoactivatable metal ligand complex is ruthenium (tris)bipyridyl chloride.

In some embodiments, the electron acceptor is sodium persulphate.

In certain embodiments, the tissue is corneal tissue.

In some embodiments, the endogenous protein is a corneal stromal protein. For example, the corneal stromal protein may be selected from collagen and/or stromal proteoglycan. In some embodiments, the corneal stromal proteins include collagens (Types I, III, V and VI) and proteoglycans (Aggrecan, Keratocan, Lumican, Mimican, Decorin and Biglycan.

In some embodiments, the step of contacting the corneal tissue with a molecular linker, a photoactivatable metal ligand complex, and an electron acceptor comprises administering a solution comprising the molecular linker, photoactivatable metal ligand complex, and electron acceptor to the surface of the cornea, whereby the solution diffuses into the corneal stroma.

In some embodiments, the step of contacting the corneal tissue with a molecular linker, a photoactivatable metal ligand complex, and an electron acceptor comprises administering two or more solutions collectively comprising the molecular linker, photoactivatable metal ligand complex, and electron acceptor to the surface of the cornea, whereby the solutions diffuse into the corneal stroma.

In some embodiments, the solution, or two or more solutions, additionally comprise high molecular weight gelatin.

In some embodiments, the tissue is irradiated with light having a wavelength of between around 430 nm to 480 nm. In certain embodiments, the tissue is irradiated with blue light having a peak wavelength of about 455 nm.

In some embodiments, irradiating the tissue comprises delivering a light intensity of between about 200 mW/cm² and 1,200 mW/cm² to the tissue surface.

In further embodiments, irradiating the tissue comprises delivering radiant energy below about 10 J/cm². For example, in some embodiments, irradiating the tissue comprises illumination of the tissue with a 455 nm light source delivering between about 1 J/cm² to about 5 J/cm². In some embodiments, irradiating the tissue comprises illumination of the tissue with a 455 nm LED light source delivering up to 3.4 J/cm².

In some embodiments, the tissue is irradiated for about 1 to about 10 seconds, or about 1 to 5 seconds.

In another aspect, there is provided a method of treating a weakened corneal stroma in a subject, the method comprising:
(i) administering a molecular linker, a photoactivatable metal ligand complex, and an electron acceptor to a cornea of the subject; and
(ii) irradiating the cornea.

The step of irradiating the cornea in the presence of the molecular linker, photoactivatable metal ligand complex, and electron acceptor, initiates a cross-linking reaction between the molecular linker and endogenous protein in the corneal stroma.

In another aspect, there is provided a method of treating or preventing at least one symptom of keratoconus in a subject, the method comprising:
(i) administering a molecular linker, a photoactivatable metal ligand complex, and an electron acceptor to a cornea of the subject; and
(ii) irradiating the cornea.

The step of irradiating the cornea in the presence of the molecular linker, photoactivatable metal ligand complex, and electron acceptor, initiates a cross-linking reaction between the molecular linker and endogenous protein in the corneal stroma.

In some embodiments, the molecular linker is a polymer comprising two or more tyrosine and/or tyramine residues.

In some embodiments, the polymer comprising two or more tyrosine and/or tyramine residues is selected from tyrosine and/or tyramine-derivatised gelatin, tyrosine and/or tyramine-derivatised dextran, and/or tyrosine and/or tyramine-derivatised xylan and/or tyrosine and/or tyramine-derivatised low molecular weight nanocellulose.

In some embodiments, the molecular linker has a molecular weight between about 5 kDa to about 40 kDa, about 10 kDa to about 20 kDa, or about 12 kDa to about 20 kDa, or about 10 kDa to about 15 kDa.

In some embodiments, the photoactivatable metal ligand complex is ruthenium (tris)bipyridyl chloride.

In some embodiments, the electron acceptor is sodium persulphate.

In some embodiments, the endogenous protein is a corneal stromal protein. For example, the corneal stromal protein may be selected from collagen and/or stromal proteoglycan. In some embodiments, the corneal stromal proteins include collagens (Types I, III, V and VI) and proteoglycans (Aggrecan, Keratocan, Lumican, Mimican, Decorin and Biglycan.

In some embodiments of the method described herein, the step of administering the molecular linker, photoactivatable metal ligand complex, and electron acceptor comprises administering a solution comprising the molecular linker, photoactivatable metal ligand complex, and electron acceptor to the surface of the cornea, whereby the solution diffuses into the corneal stroma.

In some embodiments of the method described herein, the step of administering the molecular linker, photoactivatable metal ligand complex, and electron acceptor comprises administering two or more solutions collectively comprising the molecular linker, photoactivatable metal ligand complex, and electron acceptor to the surface of the cornea, whereby the solutions diffuse into the corneal stroma.

In some embodiments, the solution diffuses into the corneal stroma for up to about 5 minutes.

In some embodiments, the cornea is irradiated with light having a wavelength of between around 430 nm to 480 nm. For example, the cornea may be irradiated with blue light from an LED light source having a peak wavelength of about 455 nm. In some embodiments, the LED light emits blue light with usable wavelength from 430 nm to 480 nm with a center wavelength of 455±10 nm. In some embodiments, the light intensity (between 400 and 515 nm) may be about 1200 mW/cm²—10%/+20% (independent of battery power level). In some embodiments, irradiating the tissue comprises illumination of the tissue with a 455 nm LED light source delivering up to 3.4 J/cm².

In one embodiment, the tissue is irradiated for about 1 to about 10 seconds, or for about 1 to about 5 seconds.

In some embodiments, cross-linking the molecular linker and endogenous protein results in an improvement in one or more mechanical properties of the corneal stroma. For example, the improvement in one or more mechanical properties of the corneal stroma may be an increase in the elastic modulus of the stroma.

In another aspect, there is provided use of a molecular linker in the manufacture of a medicament for the treatment or prevention of one or more symptoms of disease in a subject.

In some embodiments, the disease is keratoconus and/or weakening of the corneal stroma.

In another aspect, there is provided a composition for the treatment of keratoconus in a subject, the composition comprising a molecular linker, a photoactivatable metal ligand complex, and an electron acceptor.

In yet another aspect, there is provided a composition for intermolecular crosslinking between endogenous stromal collagen fibrils and other endogenous proteins in the cornea, wherein the composition comprises a molecular linker, a photoactivatable metal ligand complex, and an electron acceptor.

In some embodiments, the endogenous stromal proteins in the cornea are selected from collagen and proteoglycan.

In some embodiments of the methods described herein, the method further comprises applying high molecular weight gelatin to the surface of the cornea.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

### BRIEF DESCRIPTION OF THE FIGURES

The present disclosure can be more completely understood with reference to the following Figures.

FIG. 1. Organization of collagen and proteoglycans in corneal fibrils. Adapted from: Box 4.9—Organization of tissue proteoglycans (In: Chapter 4; Biochemistry and cell biology. Forrester, J. V., Dick, A. D., McMenamin, P. G., Roberts, F., & Pearlman, E. (2020). The Eye: basic sciences in practice (5th Edition Elsevier) (1A). Diagram of crosslinking scenario: tyr-Dextran molecular linker crosslinking within and between molecules on the collagen fibril surface, and within proteoglycan core proteins in the interfibrillar space (1B).

FIG. 2. SDS-PAGE of autoclave treated gelatin solution. 75 mg/ml BH-Nippi gelatin in PBS pH 7.4, autoclaved for the time indicated for each lane. 1: Standards; 2: Nippi gelatin; 3: BH-Nippi gelatin; 4. 20 min autoclave; 5: 40 min autoclave; 6: 60 minute autoclave; 7: 80 min autoclave; 8: 5 hour autoclave; 9: 9 hour autoclave; 10: 12 hour autoclave.

FIG. 3. Dextran functionalized or derivatised with amine groups (3A). 10 kDa Tyramine-derivatised Dextran can form dityrosine crosslinks between endogenous proteins separated by up to 25 nm of intermolecular distance (3B).

FIG. 4. Dextrans containing a primary amine at each end of the dextran polymer.

FIG. 5. Fresh bull eye, epithelium intact (5A). Debrided epithelium using an 8 mm diameter biopsy punch (5B).

FIG. 6. 25 μL of 1 mM Ru(bpy)₃ was added to the debrided zone of the cornea (6A). Frozen corneal button showing 8 mm debrided area to which 25 μL of 1 mM Ru(bpy)₃ was added (6B). Frozen cornea—1 mm thick cross section cut after Ru(bpy)₃ diffusion into the cornea for 6 minutes (6C).

FIG. 7. Visualisation of Ru(bpy)₃ fluorescence following diffusion through debrided cornea (fluorescence image captured through red filter).

FIG. 8. Riboflavin diffused into fresh bull cornea (post-blotting) (8A). Riboflavin diffusion through cornea—frozen section (8B). Visualisation of Riboflavin fluorescence following diffusion through debrided cornea (fluorescence image captured through red filter) (8C).

FIG. 9. Debride entire epithelium from cornea. Add 300 μL of 1 mM Ru(bpy)₃+50 mM SPS to anterior corneal surface: allow 10 min diffusion time (9A). Piping nozzle (metal) as mould. Irradiate for 20 sec using 450 nm blue LED at 1 cm distance (9B)

FIG. 10. Unstructured Cornea fixed into mould shape after photo-crosslinking in the presence of Ru(bpy)₃+SPS (10A). Photo-crosslinked cornea form held by forceps (10B).

FIG. 11. Cornea after crosslinking on template without SPS—No formed templated structure apparent (11A). Cornea after photochemical crosslinking without SPS—no formed templated structure apparent (11B). Forceps holding cornea after photochemical crosslinking without SPS—no formed templated structure apparent (11C).

FIG. 12. Diffusion of 0.5 mM Ru(bpy)₃ (FIG. 12A). Diffusion of 1.0 mM Ru(bpy)₃ (12B). Diffusion of 2.0 mM Ru(bpy)₃ (12C).

FIG. 13. Diffusion of 0.5 mM Ru(bpy)₃+50 mM SPS (13A); 1.0 mM Ru(bpy)₃+50 mM SPS (13B); and 2.0 mM Ru(bpy)₃+50 mM SPS (13C) into a 1 mm thick sheet of 12% gelatin.

FIG. 14. Diffusion of 2.6 mM (0.1%) Riboflavin into 12% gelatin gel over time up to 14 minutes.

FIG. 15. 12% gelatin strip crosslinked in the presence of autoclaved BH-derivatised Nippi gelatin (150 mg/ml) with 1 mM Ru(bpy)₃+50 mM SPS included in the photochemical crosslinking reaction (15A). Cantilevered gelatin strip: 0 mg mass added. 12% gelatin strip crosslinked in the presence of 7× autoclaved BH-derivatised Nippi gelatin (150 mg/ml) with 1 mM Ru(bpy)₃+50 mM SPS included in the photochemical crosslinking reaction (15B). Cantilevered gelatin strip: 150 mg mass added. 12% gelatin strip crosslinked in the presence of autoclaved BH-derivatised Nippi gelatin (150 mg/ml) with 1 mM Ru(bpy)₃+50 mM SPS included in the photochemical crosslinking reaction (15C). Cantilevered gelatin strip: 150 mg mass removed. 12% gelatin strip crosslinked in the presence of 7× autoclaved BH-derivatised Nippi gelatin (150 mg/ml) with 1 mM Ru(bpy)₃+50 mM SPS included in the photochemical crosslinking reaction (15D). 12% gelatin strip crosslinked in the presence of 1 mM Ru(bpy)₃+50 mM SPS included in the photochemical crosslinking reaction (with NO added “molecular staple”) (15E). Cantilevered gelatin strip: 0 mg mass added. 12% gelatin strip crosslinked in the presence of 1 mM Ru(bpy)₃+50 mM SPS included in the photochemical crosslinking reaction (NO “molecular staple” added) (15F). Cantilevered gelatin strip: 150 mg mass added. 12% gelatin strip crosslinked in the presence of 1 mM Ru(bpy)₃+50 mM SPS included in the photochemical crosslinking reaction (NO “molecular staple” added) (15G). Cantilevered gelatin strip: 150 mg mass removed. 12% gelatin strip crosslinked in the presence of 1 mM Ru(bpy)₃+50 mM SPS included in the photochemical crosslinking reaction (NO “molecular linker/staple” added) (15H).

FIG. 16. Fresh bovine corneal strips (10 mm×5 mm) crosslinked in the presence (16A) or absence (16B) of LMW BH-derivatised gelatin (150 mg/ml). The bovine cornea (10 mm×5 mm) was crosslinked in the presence of 7× autoclaved BH-derivatised Nippi gelatin (see lane 10, FIG. 2) (150 mg/ml) with 1 mM Ru(bpy)₃+50 mM SPS in the photochemical crosslinking reaction (total volume=60 μL).

### KEY TO THE SEQUENCE LISTING

The present application is filed together with a Sequence Listing in electronic form. The entire contents of the Sequence Listing are hereby incorporated by reference.
SEQ ID NO: 1—amino acid sequence of human Collagen Type 1A
SEQ ID NO: 2—amino acid sequence of Tyrosine-derivatised human Collagen (gelatin) Type IA
SEQ ID NO: 3—amino acid sequence of human Collagen V
SEQ ID NO: 4—amino acid sequence of human Vimentin
SEQ ID NO: 5—amino acid sequence of human Fibronectin
SEQ ID NO: 6—amino acid sequence of human Laminin
SEQ ID NO: 7—amino acid sequence of human Elastin
SEQ ID NO: 8—amino acid sequence of human Aggrecan
SEQ ID NO: 9—amino acid sequence of human Keratocan
SEQ ID NO: 10—amino acid sequence of human Lumican
SEQ ID NO: 11—amino acid sequence of human Mimican
SEQ ID NO: 12—amino acid sequence of human Decorin
SEQ ID NO: 13—amino acid sequence of human Biglycan

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Unless specifically defined otherwise, all technical and scientific terms used herein shall be taken to have the same meaning as commonly understood by one of ordinary skill in the art (for example, in protein chemistry, chemistry, cell culture, molecular genetics, molecular biology, immunohistochemistry, and biochemistry).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, typically refers to up to +/−10%, for example +/−5%, of the designated value.

As used herein, the term “subject” shall be taken to mean any animal including mammals, for example a human.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

### Selected Definitions

As used herein, the term ‘crosslinking’ refers to the process of linking two amino acids or atoms to each other via a covalent bond. One example of a crosslinking process is the reaction of a protein linking group with the sidechain residue of a protein of interest, such as an endogenous protein in a tissue, to produce a stable covalent linkage.

The term ‘molecular linker’ as used herein refers to low molecular weight polymers comprising two or more residues with tyrosine or tyramine side-chains able to form dityrosine crosslinks with tyrosine residues located on endogenous proteins in a tissue.

The term ‘photoactivatable metal-ligand complex’ as used herein refers to a metal-ligand complex in which the metal can enter an excited state when irradiated such that it can donate an electron to an electron acceptor in order to move to a higher oxidation state and thereafter extract an electron from an aromatic side chain, such as tyrosine, from tyramine or tyrosine containing protein or tyramine or tyrosine-derivatised polymer, to produce a free radical without reliance on the formation of singlet oxygen. Suitable metals include, but are not limited to, Ru(II), Pd(II), Cu(II), Ni(II), Mn(II), and Fe (III), in the form of a complex which can absorb light in the visible regions, for example an Ru(II) bipyridyl complex, a Pd(II) porphyrin complex, a sulfonatophenyl Mn(II) complex, or a Fe(III) protoporphorin complex.

As used herein, the term ‘electron acceptor’ refers to a chemical entity that accepts an electron transferred to it and so refers to an easily reduced molecule (or oxidizing agent) with a redox potential sufficiently positive to facilitate a crosslinking reaction. Electron acceptors suitable for use in the method of the present disclosure can be readily determined by the person skilled in the art using the techniques described herein. For example, in some embodiments, the electron acceptor may be a persulfate, periodate, perbromate, or perchlorate compound, vitamin B12. In one embodiment, the persulfate anion is used as the electron acceptor.

The terms ‘contacting’ or ‘administering’ as used herein refer to the application of the crosslinking reagents described herein to the surface of a tissue, for example such as corneal tissue, for a time sufficient for the reagents to diffuse into the tissue. One or more of the crosslinking reagents may be applied to the surface of the tissue sequentially, in any order, or simultaneously, such as in a single solution comprising one or more of the molecular linker, photoactivatable metal ligand complex, and/or the electron acceptor.

As used herein, the terms “polypeptide” and “protein” are used interchangeably. The term “polypeptide” also includes post translational modified polypeptides or proteins. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 9, 10, 20, 30 or 50 amino acids. In some embodiments, peptides are between 5 and 30 amino acids in length. A peptide may be made by protease digestion of a large polypeptide.

The term “biocompatible” as used herein, means causing minimal or no clinically relevant tissue irritation, injury, toxic reaction, or immunologic reaction to human tissue based on a clinical risk/benefit assessment.

### Molecular Linkers

The present inventors have developed molecular linkers useful for rapid formation of intermolecular crosslinks between endogenous protein molecules within a tissue. These molecular linkers (also referred to herein as ‘molecular staples’) effectively bridge, or span, degraded regions of endogenous proteins in a tissue, or tissues, that have in consequence, been weakened. The molecular linkers, for example tyrosine-derivatised low molecular weight linkers, may be selected from low molecular weight polymers comprising side-chains able to form π-π hydrophobic interactions with tyrosine residues located on endogenous stromal proteins.

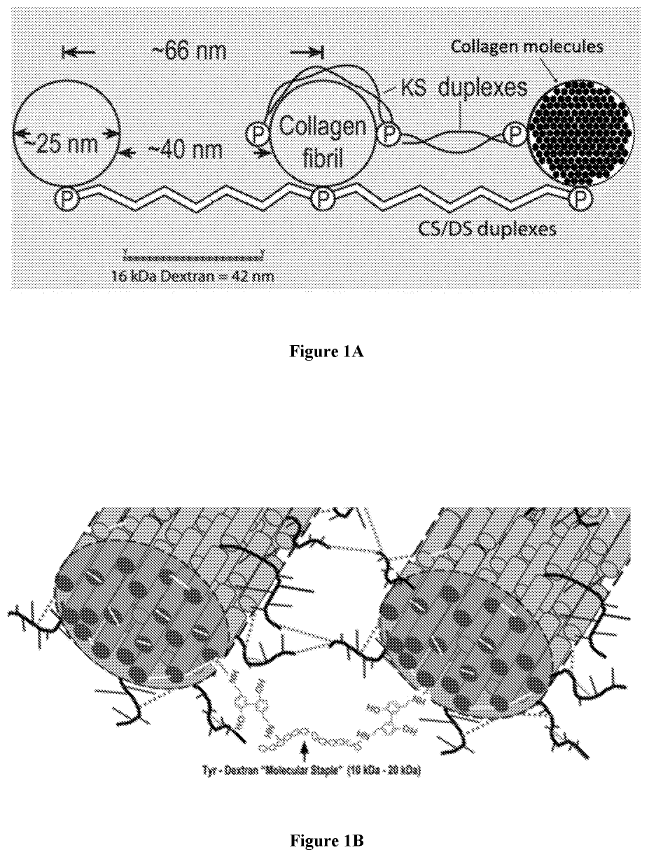

The length of the molecular linkers enables formation of covalent crosslinks between endogenous proteins that are separated by molecular spaces greater than can be achieved with currently available methods, such as the riboflavin/UV-A based CXL methods, which act only at short distances (<5 Å). While the current CXL method relies on the generation of short-lived, short-range, diffusible singlet oxygen radicals, the rapid photochemical method described herein permits formation of crosslinks between the molecular linker polymer and two or more distantly separated endogenous proteins. Thus, the molecular linkers described herein can be covalently crosslinked to join distantly separated molecules of endogenous proteins, such as, by way of non-limiting example, separated collagen molecules located on separate collagen fibrils, forming interfibrillar collagen crosslinks. FIG. 1 depicts possible collagen fibril crosslinking scenarios with a tyr-derivatised dextran molecular linker. The molecular linkers described herein have been designed by the present inventors to be of a molecular weight sufficiently low so as to allow the linkers to diffuse into a tissue, at a clinically useful rate.

### Diffusion of Polymers Through the Corneal Stroma

One exemplary tissue into which the molecular linkers can diffuse freely is the corneal stroma. The corneal stroma has a relatively open “gel-like” structure allowing the diffusion of compounds with a molecular weight below 500,000 Da, although the actual molecular weight may be much lower (Prausnitz and Noonan, 1998). The negatively charged hydrophilic corneal stroma forms a weak barrier for lipophilic drug penetration.

Rajapaksha et al., (2015) investigated the rate of diffusive transport in excised porcine corneal stroma using fluorescently labeled dextran molecules with hydrodynamic radii ranging from 1.3 nm to 34 nm (representing molecular weights ranging from 3 kDa to 2,000 kDa). They found that even dextrans of molecular weight approximately equal to 2,000 kDa can penetrate through 30% of the stromal thickness in 30 min, compared to the diffusion distance recorded for the low molecular weight fluorophore molecule fluorescein (MW=332 Da). A LMW fluorescein-labelled dextran (MW≈3.000 Da) diffused through the stroma at 70% of the rate of free fluorescein (Cui et al., 2011). The corneal stroma is therefore a relatively leaky extracellular matrix, allowing free diffusion of low molecular weight proteins, including the tyrosine- (or tyramine) derivatised molecular linkers described herein.

The present inventors have determined that any low molecular weight, biocompatible polymer comprising residues with side-chains capable of photochemical crosslinking with tyrosine may be suitable for use in the crosslinking method described herein. To ensure free diffusion of the molecular linkers into the corneal stromal matrix, the molecular weight of these polymers should be of a size that enables their free diffusion into and through the stroma within a practically useful short period of time—measured in minutes. The person skilled in the art can readily determine time taken for a polymer of a given molecular weight to diffuse into the corneal stroma using methods as described herein. Examples of suitable low molecular weight polymers include size-selected gelatin, dextrans, xylans or low molecular weight nanocellulose. Preferably, the polymers used as molecular linkers have a molecular weight of between about 5 kDa to about 100 kDa, or about 10 kDa to about 60 kDa, or about 10 to about 50 kDa, or about 10 kDa to about 20 kDa, or about 12 kDa to about 20 kDa, or about 10 kDa to about 15 kDa, or alternatively about 5 kDa, about 10 kDa, about 15 kDa, about 20 kDa, about 25 kDa, about 30 kDa, about 35 kDa, or about 40, 50, 60, 70, 80, 90, or 100 kDa.

### Synthesis of Molecular Linkers

Molecular linkers suitable for use in the crosslinking method described herein comprise at least two residues with side-chains capable of crosslinking with tyrosine residues in endogenous proteins in a tissue through formation of covalent dityrosine bonds. By way of example, the molecular linker may be a peptide or protein polymer containing two or more tyrosine, or tyramine residues capable of interacting and crosslinking with amino acid residues in endogenous proteins using the method described herein. In one embodiment, the molecular linker is size-selected gelatin, for example, tyramine-derivatised gelatin. In other embodiments, the molecular linker may be another biocompatible polymer, such as, but not limited to, dextran, xylan or low molecular weight nanocellulose. Other tyramine-derivatised carbohydrate molecules, such as chitosan and PVA can also be photochemically crosslinked into polymeric hydrogels, through formation of dityrosine covalent bonds.

The skilled person will understand that, depending on the chosen polymer, it may be necessary to perform a method of size selection to obtain polymers of a suitable length/molecular weight for use in the crosslinking method described herein.

In addition, the present inventors consider that increasing the number, or density, of residues capable of crosslinking with endogenous proteins improves the crosslinking method by resulting in a higher covalent crosslink density in the tissue. Methods of chemical derivatisation for increasing the number of crosslinkable tyrosine or tyramine residues in a polymer are known in the art.

### Size Selected Tyrosine-Derivatised Gelatin

In some embodiments, the molecular linker comprises size selected tyrosine-derivatised gelatin. For example, Bolton-Hunter derivatised, pharmaceutical grade gelatin may be used as a molecular linker to enhance stromal protein crosslinking. Type B porcine gelatin (Type 1 gelatin) may be chemically derivatised (using Bolton-Hunter reagent) to replace up to 38 Lysine (K) amino acid residues with tyrosine residues, as described (Elvin et al., 2010). In this regard, gelatin may be derivatised to increase its Tyr content, using Bolton-Hunter reagent (N-succinimidyl-3-[4-(hydroxy)propionate), as described (Bolton and Hunter, 1973; Miller, 1996), with slight modification. A 10% solution of Nippi Inc. Medi-Gelatin can be dissolved in sodium borate/saline buffer pH 8.6 and reacted with Bolton-Hunter reagent (Sigma) in a 25:1 (w:w) ratio at 37° C. for 1 h. This reaction mixture is extensively dialysed against PBS at 37° C. and subsequently freeze-dried. This derivatisation process increases the tyrosine content of gelatin from 4/1,000 residues for native gelatin (0.4 mol %) to 38/1,000 residues (3.8 mol %) for Bolton-Hunter derivatised gelatin. The average molecular weight (MW) of the BH-derivatised gelatin is about 100 kDa, with strong bands visible on SDS-PAGE at 100 kDa (α-bands), 200 kDa (β-bands) and 300 kDa (γ-bands). For production of gelatin-based molecular linkers, the average molecular weight of the derivatised gelatin should, preferably, be reduced to a size range of 10 kDa to 20 kDa. There are several approaches to reduce the average MW of gelatin, including repeated cycles of either autoclaving or sonication.

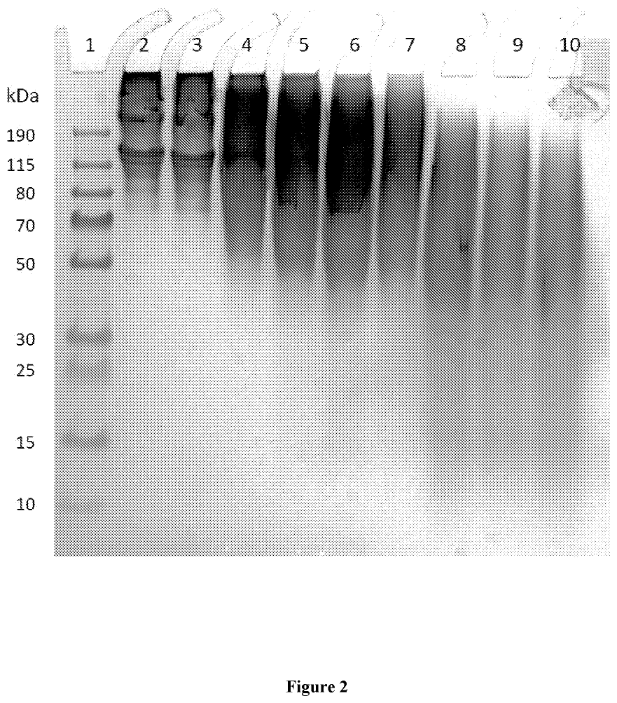

As described by Vanhoecke and Olijve, 2018, autoclave treatment of gelatin solution can be performed to decrease the molecular weight of the gelatin. For example, a 10% solution of a type A gelatin from Sigma-Aldrich (G1890) can be autoclave sterilized for 30 minutes at 121° C., resulting in a reduction of MW from 150 kDa to about 70 kDa. Furthermore, increased autoclave time cause a greater level of degradation of gelatin solutions, resulting in production of smaller molecular weight gelatin fragments. The present inventors have demonstrated that a 150 mg/ml (15% w/v) solution of Bolton-Hunter derivatised Nippi MediGelatin was degraded to fragments of MWₐᵥₑ=15 kDa following up to 14 hours of autoclaving (121° C.) (see SDS-PAGE analysis, FIG. 2). This molecular weight range of polymers is capable of almost unhindered diffusion through the corneal stroma.

Another approach for preparing low molecular weight gelatin fraction is ultrasonication (Kanegae et al., 1992). The starting material may be a high molecular weight sample of gelatin (for example, Type B gelatin derived from bone) and may comprise a range of molecular weight gelatin fractions, including γ- (~300 kDa), β- (~200 kDa) and α- (100 kDa). Sonication results in a faster rate of degradation of high molecular weight gelatin fractions (γ-, β-) while lower molecular weight fractions (α- and smaller fragments) are degraded at a slower rate. For example, 4 minutes of sonication at a frequency of 19.5 kHz with an intensity of 6V is required to degrade a 7.5% gelatin solution of gamma fraction (γ=300 kDa) to low molecular weight (Mₐᵥₑ=40 kDa) gelatin molecules; 8 minutes of sonication was required to degrade the beta fraction (β=200 kDa) to low molecular weight (Mₐᵥₑ=40 kDa) gelatin molecules; and 16 minutes of sonication was required to degrade alpha fraction (α=100 kDa) to low molecular weight (Mₐᵥₑ=40 kDa) gelatin molecules. On this basis, in order to prepare a 10 kDa to 20 kDa sample of degraded Nippi MediGelatin (that comprises relative size fraction abundance of γ:β:α of 1:1:2), a person skilled in the art would sonicate a 7.5% solution of high molecular weight gelatin dissolved in deionised water, for approximately 30 minutes maintained in a water bath at 20° C.

The skilled person will appreciate, in light of the teachings herein, that a size-selected sample of derivatised gelatin may contain molecular linkers in a range of molecular weights, for example from around 5 kDa to around 60 kDa, or around 10 kDa to around 50 kDa, depending on the method used for the preparation of the size-selected molecular linkers. Preferably, a portion of the molecular linkers are in the molecular weight range of about 5 kDa to about 20 kDa, or about 10 kDa to about 15 kDa.

An example amino acid sequence of Tyrosine-derivatised human Collagen (gelatin) Type I is provided in SEQ ID NO: 2.

### Non-Degradable (Protease Insensitive) Molecular Linkers

There are several approaches for preparing suitable non-degradable molecular linkers (‘molecular staples’), that are not susceptible to proteolysis, for crosslinking of endogenous proteins in a tissue. These include carbohydrate-based polymers such as Dextrans, Xylans and Nanocellulose. Dextrans are biopolymers authorised by the FDA for us in ocular eye drops and as treatment for dry eye. They are comprised of α-1,6 linked D-glucopyranose residues with a few percent of α-1,2, α-1,3, or α-1,4-linked side chains.

Xylans are polysaccharides made up of β-1,4-linked xylose residues with side branches of α-arabinofuranose and/or α-glucuronic acids; Xylan (Mw=12.7 kDa, 20 kDa)—is a natural hemicellulose polysaccharide composed of arabinose, 4-O-methyl-glucuronic acid (10 wt %) and xylose in a ratio of 1:2:11 respectively (Escalante et al., 2012).

Cellulose is a plant-derived polysaccharide polymer consisting of chains of β-(1-4)-linked-D-glucose repeating units assembled into nanosized, thread-like agglomerates called micro-fibrils.

Non-limiting examples of different classes of biocompatible, non-degradable, protease-insensitive polymers as starting materials for the synthesis of molecular linkers suitable for photochemical crosslinking of endogenous corneal stromal proteins, include:
(i) LMW tyrosine-derivatised Dextran (10 kDa and 20 kDa);
(ii) LMW Bis-tyrosine derivatised Dextran (10 kDa and 20 kDa);
(iii) LMW tyramine-derivatised Xylan (12.7 kDa and 20 kDa); and
(iv) LMW Tyramine-derivatised nanocellulose (10 kDa-20 kDa).

### Dextrans

Dextran polymers with Mw<40 kDa can be eliminated through renal clearance and have a half-life of 8 h, whereas molecules with Mw>40 kDa have larger half-lives and would be sequestered in the liver and spleen and then hydrolysed by endo- and exo-dextranases. Biodegradation occurs through natural enzyimatic splitting of saccharide bonds by dextran-1,6-glucosidase found in spleen, liver, lungs, kidneys, brain, and muscle tissue as well as by dextranases expressed by bacteria in the colon.

### (i) Tyrosine-Derivatised Amino Dextrans

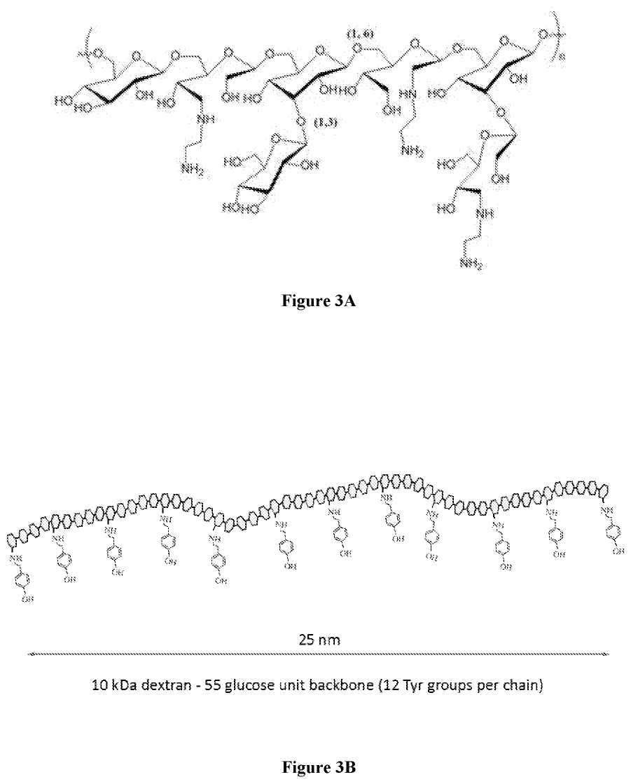

The person skilled in the art can readily obtain amino-functionalised dextrans. For example, amino-functionalised dextrans are commercially available from FinaBioSolutions. A 10 kDa amino-dextran, with 10 primary amine-groups per polymer and a 20 kDa amino-dextran product with 20 primary amine-groups per polymer are suitable candidates, with their low molecular weights allowing free diffusion through the corneal stroma. Tyrosine groups can be added to the reactive primary amine-groups, using the —NH₂ reactive Bolton-Hunter method, as described by Elvin et al., (2010). The tyrosine-derivatised dextrans and BIS-dextrans can be purified using methods known in the art, such as by ethanol precipitation using the method described by Xu et al., (2014). FIG. 3A shows a dextran polymer derivatised with amine groups. FIG. 3B shows a 10 kDa Tyramine-derivatised Dextran that is able to form dityrosine crosslinks between endogenous proteins separated by up to 25 nm of intermolecular distance. Given that a single glucose molecule is about 1 nm in length, a 20 kDa Tyramine-derivatised Dextran molecular linker can form dityrosine crosslinks between endogenous proteins separated by up to 50 nm of intermolecular distance.

### (ii) Tyrosine-Derivatised Bis-Amino Dextrans

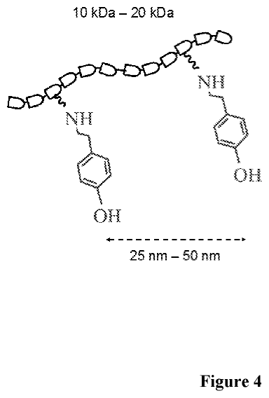

The skilled person will understand that di-amino dextrans are also suitable for use as molecular linkers in the method described herein. Di-amino dextrans are commercially available from FinaBioSolutions. The dextrans contain a primary amine at each end of the dextran polymer (FIG. 4). Both the 10 kDa and the 20 kDa bis-amino dextrans can be derivatised using the Bolton-Hunter method described by Elvin et al., (2010). The tyramine-derivatised dextrans may then be purified by ethanol precipitation in order to remove non-dextran reagents, as described in Xu et al. (2014). For dextran polymers of around 20 kDa, an ethanol concentration of about 90% is required for quantitative precipitation of the derivatised dextran.

(iii) Tyramine-Derivatised Xylans

Xylans are polysaccharides made up of β-1,4-linked xylose residues with side branches of α-arabinofuranose and/or α-glucuronic acids. Xylan (Mw=12.7 kDa, 20 kDa) —natural hemicellulose polysaccharide composed of arabinose, 4-O-methyl-glucuronic acid (10 wt %) and xylose in a ratio of 1:2:11 respectively (Escalante et al., 2012). Xylan remains undigested in human tissues and can only be degraded by anaerobic colon microflora. Xylan is non-cytotoxic and biocompatible—xylan-based hydrogels were shown to be non-cytotoxic. (Khaire et al., 2022). Tyr-derivatised xylan is soluble in water to 40% (w/v) concentration (Kuzmenko et al., 2014).

It has been shown previously that hemicelluloses extracted from spruce, such as xylan and galactoglucomannan, could be modified with tyramine, a molecule similar to lignin, which enabled subsequent cross-linking of the aromatic residues using the enzyme horseradish peroxidase. (Kuzmenko et al., 2014; Markstedt et al., 2017).

Xylan isolated from spruce was successfully conjugated with tyramine using EDAC/NHS activation of glucuronic acid in spruce arabinoglucuronoxylan. This conjugate was enzymatically crosslinked with HRP to provide gelation within 20±5 s at room temperature. Hydrogels showed mechanical integrity and an interconnected porous structure, as well as high degree of swelling. This system was used for in situ immobilized mesenchymal stem cells, and the cells were able to differentiate into adipocytes, with no evidence of cytotoxicity. Previous in vivo studies showed that hydrogels crosslinked in 100 mM H₂O₂ are biocompatible and non-toxic.

### (iv) Tyramine-Derivatised Nanocellulose

Cellulose and its derivatives have been utilized extensively as textile fibers, chemical precursors and for paper making and food additives. Cellulose whiskers (nanocrystals), obtained by acid hydrolysis of cellulose, are a more recent area of application for nanocomposites. Acid hydrolysis yields crystalline rods or whisker-shaped nanoparticles with diameters that range from 8 to 20 nm and lengths of 100 nm to few micrometers, depending on the source of the cellulose (Habibi et al., 2010; Lima et al. 2004). Such whiskers have high elastic modulus, high aspect ratio, and their surface chemistry can be modified to broaden their use in high-value applications.

Chemical modifications of cellulose nanocrystals and nanofibres are known in the art and include esterification, cationization, carboxylation, silylation and polymer grafting. Most of these techniques use the abundance of hydroxyl groups on the surface to facilitate the easy conjugation of desired molecules.

Nanocellulose fibres of the desired molecular weight (for example, from about 10 kDa to about 20 kDa) and length (for example, from about 50 nm to about 100 nm long nanofibers) may be produced using water at high temperature and pressure (supercritical water) to degrade and extract short low molecular weight cellulose nanofibers (Buffiere, et al., 2016).

Nanocellulose fibres and crystals of the desired molecular weight and size are biocompatible and safe to cells in vitro and to animals in vivo. Nanocellulose represents a promising biomaterial for tissue regeneration due to its favourable biocompatibility, and relatively low toxicity. A number of independent studies have described the low toxicity and biocompatibility of cellulose nanoparticles and fibres.

A number of chemical synthetic methods, known to those skilled in the art, can be used to prepare amino-functionalised nanocellulose fibres. Such amine-derivatised nanocellulose whiskers may be prepared as aqueous suspensions at 20% (w/v). Synthetic chemical approaches to introduce amino-reactive groups into cellulose polymers include methods described by Shaghaleh et al. (2021), Heinze et al. (2016) and Jardine (2022).

Once amine-derivatised nanocellulose fibres are prepared, conversion of such nanofibers to the desired tyramine-derivatised form is carried out using synthetic methods described in the literature. These include use of the Bolton-Hunter reagent to convert primary amine groups to the tyramine derivative, as described (Bolton, AE, Hunter, W M., 1973).

Other approaches to synthesis of tyramine-derivatised cellulose have also been described, including the synthesis of cellulose acetoacetates (CAA) as a precursor for preparing tyramine-cellulose derivatives.

An alternative method for synthesis of tyramine-derivatised cellulose was described by Simon et al., (2023), who used the reductive amination of dialdehyde cellulose (DAC) with 2-picoline borane to generate a range of bioderived thermoplastics—the primary amine tyramine, was thereby introduced into the cellulose backbone.

Photochemical Crosslinking with Molecular Linkers

In contrast to the current CXL method, the present inventors describe herein, given the composition and structure of the corneal stroma, the molecular dimensions of this specialised extra cellular matrix (ECM) and the nature of its component protein species, the design and synthesis of a number of different molecular linkers useful for improving the strength of a tissue via photochemical crosslinking. The short (low molecular weight) polymer reagents proposed allow the formation of covalent crosslinks between distantly separated endogenous proteins. In corneal stroma tissues, such crosslinks cannot be created using the traditional riboflavin/UV-A Dresden protocol. The present inventors have determined that the crosslinking procedure described herein produces crosslinks between previously uncross-linkable stromal proteins because the proteins are physically separated by distances exceeding the capacity of the traditional CXL method to form covalent crosslinks between them. The molecular linker-mediated covalent crosslinking of stromal collagens and proteoglycans, as described herein, results in a mechanically stiffer (higher elastic modulus) ECM material, compared to the traditional CXL method.

The photochemical crosslinking method described herein may avoid one or more problems associated with the current CXL methods, due both to the chemical components of the visible-light photocrosslinking method and the wavelength and energy of blue light illumination. The method described herein involves the direct photochemical crosslinking of tyrosine residues, and to a lesser extent, histidine residues, located on various endogenous stromal ECM proteins. Example amino acid sequence of the major endogenous stromal ECM proteins are provided in SEQ ID Nos: 1 and 3 to 13. These proteins include Collagens Type I and V, Vimentin, Fibronectin, Laminin, Elastin, Aggrecan, Keratocan, Lumican, Mimican, Decorin, and Biglycan, all of which contain multiple tyrosine residues amenable to photochemical crosslinking in the method described herein.

The presently described crosslinking method avoids the production of damaging singlet oxygen free radicals. Furthermore, the efficiency of this process is high, requiring only a few seconds of blue light illumination to drive quantitative crosslinking of hydrophobically-associated proteins in the corneal stroma. The diffusion of low molecular weight ‘molecular linker’ polymers into the corneal stroma allows crosslinking of stromal collagen protein chains located up to 55 nm away from neighbouring protein chains. In this manner, degraded interfibrillar collagen stromal protein chains can be bridged by a covalently crosslinked intact “molecular linker”. See FIG. 1A, showing the arrangement of collagen molecules within fibrils and the spacing between fibrils. Note that a 16 kDa tyramine-derivatised dextran-based “molecular linker” (molecular length=42 nm) is capable of spanning the distance (about 40 nm) between 2 adjacent collagen fibrils and can therefore form crosslinks between collagen molecules located on adjacent collagen fibrils.

Given the molecular dimensions of the molecular linkers, and without wishing to be limited by theory, collagen molecules located on individual but adjacent collagen fibril bundles could become physically crosslinked, with the molecular linker serving as a bridge. In contrast, the current riboflavin/UV-A induced CXL method is a crosslinking method that can only induce crosslinking between individual collagen chains located within a collagen fibril. This limitation applies because the singlet oxygen-mediated mechanism for this free radical crosslinking method acts at only very short (<5 Å) molecular distances, allowing crosslinking of hydrophobically-associated tyrosine residues located on adjacent triplet-chain collagen molecules within the same collagen fibril.

The present inventors have demonstrated photochemical crosslinking of endogenous proteins via treatment with a solution comprising a molecular linker, a photoactivatable metal ligand complex, and an electron acceptor, and that this photochemical crosslinking can cause stable readjustment of the stromal matrix protein 3D structure in the cornea. This suggests the collagen and/or other stromal ECM proteins have become covalently crosslinked via hydrophobic molecular interactions involving tyrosine residues located on both the endogenous stromal proteins (collagen and/or proteoglycans) and the tyramine-derivatised molecular linker. That the method described herein can form new molecular associations induced by external physical stress/strain forces imparted during templating of the corneal stroma, as described in the present Examples, suggests that endogenous physical interactions must occur between tyrosine/histidine/tryptophan residues located in the various stromal matrix proteins comprising the ECM.

During the 3-D structure/templating process described in the Examples, these new molecular associations are permanently “fixed” through new covalent bond formation occurring during the brief, for example 1 to 5 seconds, photochemical crosslinking reaction. Importantly, given the capacity of the molecular linker to span up to 55 nm of intermolecular distance, new intermolecular covalent bonds can be formed between collagen proteins located on separate collagen fibrils.

The photochemical crosslinking method of the present disclosure provides for short (for example, less than about 5 seconds) illumination of a tissue, such as the cornea, using a light source (for example a 455 nm blue LED dental curing lamp, 1,200 mW/cm²) following a 3 minute period to allow diffusion of the photochemical components into the corneal stromal matrix, including the molecular linker, into the corneal stromal matrix.

Thus, steps of the crosslinking method comprise:
(i) applying the photochemical components comprising the molecular linkers to the surface of the tissue;
(ii) allowing the photochemical components to diffuse into the tissue; and
(iii) irradiating the tissue with light of a suitable wavelength and energy to facilitate crosslinking of the molecular linkers to endogenous proteins.

The use of a short illumination time with visible blue light represents a safer alternative to the potentially harmful use of UV light for traditional CXL procedures.

The skilled person will appreciate that it in some embodiments it is desirable that the crosslinking reagents diffuse no more than about halfway through the thickness of the cornea, or no more than about 300 μm, or no more than about 250 μm, or no more than about 200 μm through the cornea. The skilled person can determine suitable diffusion times based on information provided herein.

### Safety of Blue Light (455 nm) Photochemical Curing

The present inventors estimate the blue light irradiation requirement for the molecular linker crosslinking method is:
≤5 seconds* at 400 mW/cm² delivered at 2 cm from target (≡2 J/cm²) via 3M ESPE S10 LED curing light (430 nm-480 nm, peak wavelength 455 nm±10 nm, 1,200 mW/cm² at source).

Regarding the safety of blue light illumination, the method described herein falls within the current ISO standard (ISO 15004-2) which allows up to 10 J/cm² of visible light illumination to be delivered to the retina (Sliney et al., 2005).

The current Riboflavin-based CXL methods deliver 5.4 J/cm² of UV-A light (illumination at 370 nm). Recent studies by Ziółkowska, et al., (2023) demonstrated safe ophthalmic levels up to 3.4 J/cm² for retinal ganglion cells in rats exposed to LED light (463±10 nm). The proposed blue light exposure (1-5 sec of 455±10 nm blue LED light source—using the 3M ESPE S10 LED curing lamp at 50 mm distance from the cornea) described herein is equivalent to delivery of 0.4 J/cm²-2.0 J/cm².

Blue light does not generate the type of DNA damage caused by UV light. One cause of cytotoxicity with blue light photocuring relates to singlet oxygen free-radical formation. The photochemical crosslinking method described herein, however, does not generate singlet oxygen formation during the photochemical crosslinking reaction in the presence of persulphate. In addition, illumination of the cornea for 10 seconds, from a distance of 10 mm, using this LED source would deliver 2.55 J/cm² of energy (well below the 10 J/cm² defined by the ISO 15004-2 standard) (Sliney et al., 2005).

The present inventors have further determined that, in some embodiments, a lower dose of energy (<1 J/cm²) can be delivered using a 3M dental curing light with illuminance of 400 mW/cm² at 20 mm distance from the light guide tip, by reducing illumination time to about 2.5 seconds. In some embodiments, crosslinking can be achieved in 1 second of illumination at a distance of 50 mm.

### Therapeutic Methods and Compositions

The photochemical crosslinking method described by the present inventors can be used for preventative and therapeutic purposes where the improvement of the mechanical properties and/or strength of a tissues is required.

Accordingly, the present disclosure provides a method of crosslinking endogenous protein in a tissue, the method comprising:
(i) contacting the tissue with a molecular linker, a photoactivatable metal ligand complex, and an electron acceptor, whereby the molecular linker contacts the endogenous protein in the tissue; and
(ii) irradiating the tissue to cross-link the molecular linker.

By irradiating the tissue in the presence of these reagents, the molecular linker and the endogenous protein are covalently crosslinked.

In some embodiments, this method may be used in the treatment or prevention of ectatic corneal disease (ECD), or corneal ectasia, which is a group of progressive eye disorders characterized by progressive corneal steepening and thinning. In some embodiments, the method is used for the treatment or prevention of at least one symptom of keratoconus in a subject.

Thus, the present disclosure further provides a method of treating a weakened corneal stroma in a subject, the method comprising:
(i) administering a molecular linker, a photoactivatable metal ligand complex, and an electron acceptor to a cornea of the subject; and
(ii) irradiating the cornea.

Irradiating the cornea may be performed using a blue LED light curing lamp. The proposed blue light exposure (1-5 sec of 455±10 nm blue LED light source—using the 3M ESPE S10 LED curing lamp at 50 mm distance from the cornea) described herein is equivalent to delivery of 0.4 J/cm²-2.0 J/cm².

The step of irradiating the cornea in the presence of the molecular linker, photoactivatable metal ligand complex, and electron acceptor, initiates a covalent cross-linking reaction between the molecular linker and an endogenous protein in the cornea.

The present disclosure further provides a method of treating or preventing at least one symptom of keratoconus in a subject, the method comprising:
(i) administering a molecular linker, a photoactivatable metal ligand complex, and an electron acceptor to a cornea of the subject; and
(ii) irradiating the cornea.

The step of irradiating the cornea in the presence of the molecular linker, photoactivatable metal ligand complex, and electron acceptor, initiates a cross-linking reaction between the molecular linker and an endogenous protein in the cornea.

The reagents used in the therapeutic may be applied to the surface of the tissue, for example a cornea, separately in any order, or one or more reagents may be contained in a single solution for application to the tissue.

The present disclosure further provides therapeutic and/or pharmaceutical compositions comprising a molecular linker as defined herein. Further provided are therapeutic or pharmaceutical compositions comprising one or more of the molecular linker and: a photoactivatable metal ligand complex, and/or an electron acceptor.

The present disclosure further provides kits comprising the molecular linker and one or more of: a photoactivatable metal ligand complex, and/or an electron acceptor.

The person skilled in the art can readily determine suitable pharmaceutically acceptable buffers, excipients and carriers in which to prepare the crosslinking reagents. A wide variety of pharmaceutically acceptable excipients and carriers are known in the art. Such pharmaceutical carriers and excipients as well as suitable pharmaceutical formulations have been amply described in a variety of publications (see for example “Pharmaceutical Formulation Development of Peptides and Proteins”, Frokjaer et. al.., Taylor & Francis (2000) or “Handbook of Pharmaceutical Excipients”, 3rd edition, Kibbe et al., Pharmaceutical Press (2000) A. Gennaro (2000) “Remington: The Science and Practice of Pharmacy”, 20th edition, Lippincott, Williams, & Wlkins; Pharmaceutical Dosage Forms and Drug Delivery Systems (1999) H. C. Ansel et al., eds 7th ed., Lippincott, Wiliams, & Wlkins; and Handbook of Pharmaceutical Excipients (2000) A. H. Kibbe et al., eds., 3rd ed. Amer. Pharmaceutical Assoc).

In some embodiments, addition of a high molecular weight gelatin component (for example, Nippi MediGelatin) to the formulations described herein will seal the exposed corneal surface following epithelial debridement. This photocured hydrogel will enhance regrowth and healing of the epithelium as well as providing a delivery method for antibiotics, anti-inflammatory and/or pain medication to the wounded corneal surface. The use of this high molecular weight hydrogel treatment will improve patient comfort and reduce the incidence of keratitis and corneal perforations, which may occur rarely following “epi-off” surgical methods.

### EXAMPLES

The present disclosure is not to be limited in scope by the specific examples described herein, which are intended for the purpose of exemplification only. Functionally-equivalent products, compositions and methods are clearly within the scope of the invention, as described herein.

### Example 1. Bull eye diffusion study

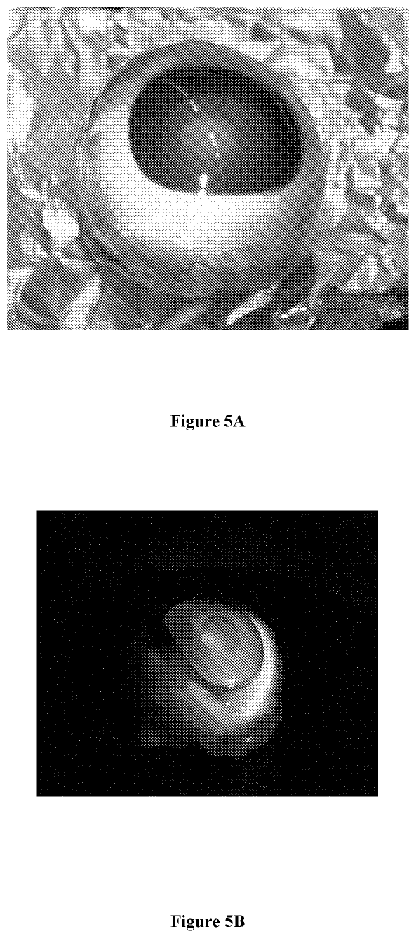

10 fresh bull eyes were obtained from an abattoir (<10 hours old, stored at 4° C.). Using a digital caliper, the thickness of the corneas was determined to be within the range of 740 μm to 820 μm. The epithelium of the eyes was debrided with an 8 mm biopsy punch and scraped-off with a scalpel blade. A fresh bull eye is shown in FIG. 5A and a bull eye with debrided epithelium is shown in FIG. 5B.

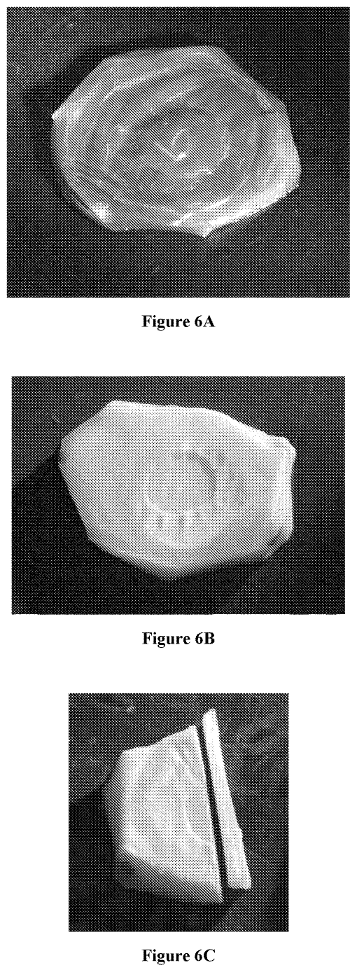

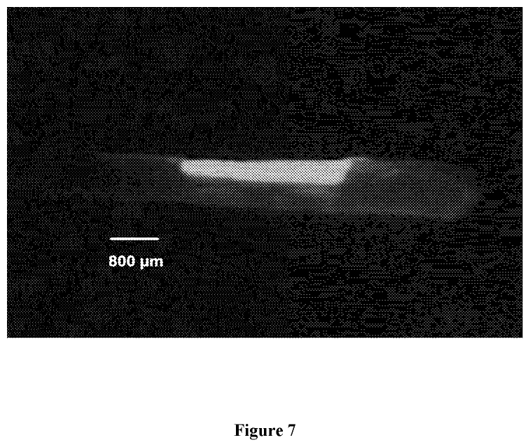

25 μL of 1 mM Ru(bpy)₃ was added to the debrided zone of the cornea (FIG. 6A) and allowed to diffuse into the cornea for 6 minutes. Excess Ru(bpy)₃ solution was removed by blotting using a fresh Kimwipe tissue.

Corneas were frozen on the surface of an aluminium block at −20° C. for 2 minutes (FIG. 7B). Transverse (bisecting) cross-sections of cornea were prepared by cutting a 1 mm thick section using a razor blade. The corneal cross-section was mounted on a glass slide to allow visualization of the cross-sectional area of the corneal stroma (FIG. 7C). Ru(bpy)₃ was visualized through a red filter with illumination with a 450 nm LED light (FIG. 7).

### Example 2. Riboflavin Diffusion (0.1% [2.6 mM] in pH 8.5 PBS)

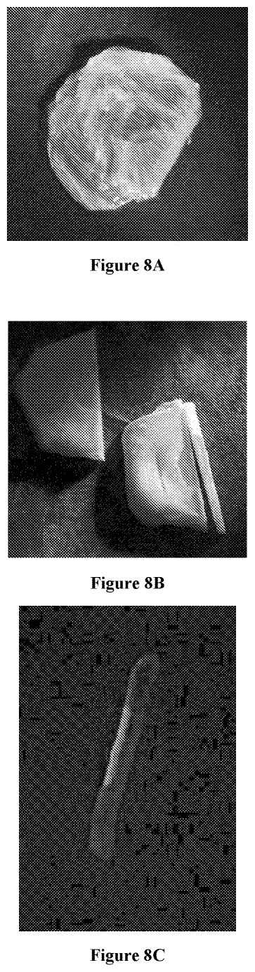

Debrided cornea was prepared from fresh bull eye as described in Example 1. 25 μL of 0.1% Riboflavin was allowed to diffuse into the debrided cornea for 5 minutes, and then excess liquid blotted (FIG. 8A).

The cornea was frozen as described in Example 1 and cut into a 1 mm thick transverse cross-section (FIG. 8B). Riboflavin fluorescence was visualized under red light filter (illumination using a 450 nm blue LED) (FIG. 8C).

### Example 3. Photochemical Crosslinking of Corneal Stroma—Using 3D Template Mould

A. Cornea Crosslinking with 1 mM Ru(Bpy)₃+50 mM Sodium Persulphate (SPS).

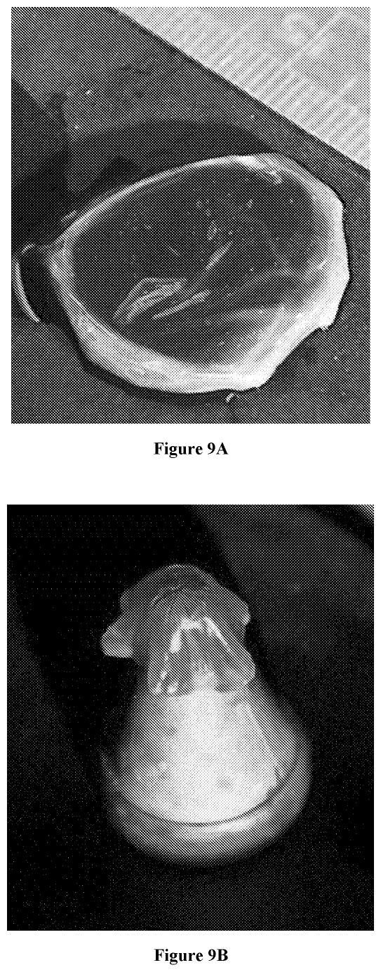

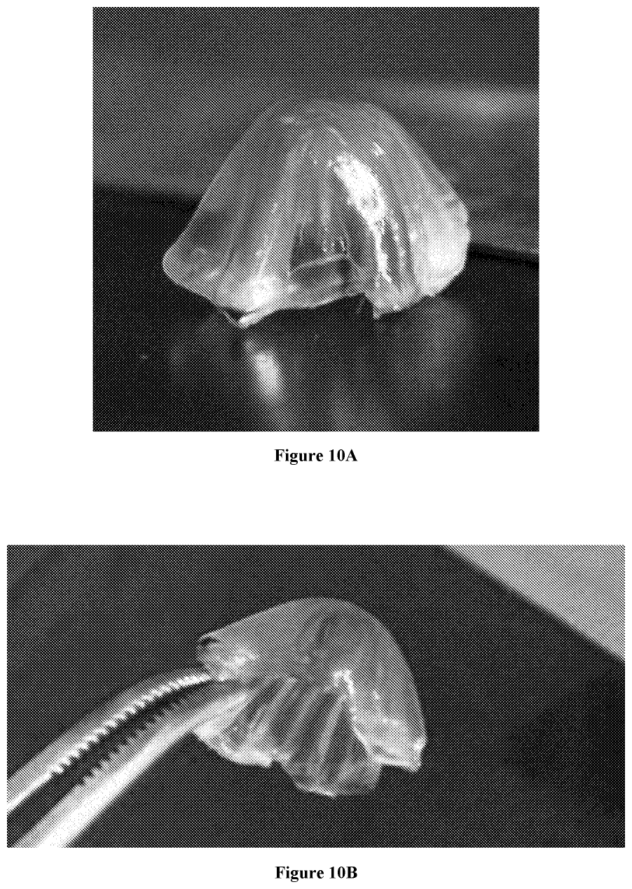

The entire epithelium was debrided from a cornea. 300 μL of 1 mM Ru(bpy)₃+50 mM SPS was applied to the anterior corneal surface for a diffusion time of 10 minutes (FIG. 9A). The cornea was wrapped over a metal piping nozzle, acting as a mould, and irradiated for 20 seconds using 450 nm blue LED dental lamp at a distance of 1 cm from the light guide tip (FIG. 9B). Post photo-crosslinking, the cornea was fixed into the shape of the mould and able to support its weight when held by forceps (FIGS. 10A and 10B).

B. Cornea Crosslinking with 1 mM Ru(Bpy)₃ ONLY (No SPS).

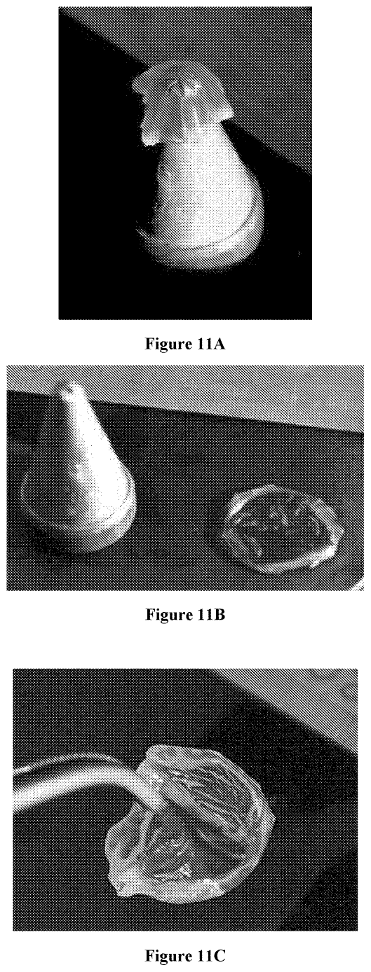

A cornea was subject to the crosslinking protocol described in Example 3A, except that sodium persulphate (SPS) was omitted. Following irradiation with 450 nm blue LED light, no mould template structure was formed and the cornea remained unstructured and malleable (FIGS. 11A to 11C). This experiment demonstrates that an electron acceptor, such as SPS, plays an important role in the photochemical formation of dityrosine covalent bonds between endogenous corneal proteins. In its absence, singlet oxygen radical production by short (20 sec) blue light irradiation of Ru(bpy)₃ in aqueous solution is insufficient to cause any measurable covalent crosslinking of endogenous stromal proteins. In contrast, in its presence, crosslinking of endogenous corneal proteins and “molecular linkers” occurs rapidly and quantitatively.

### Example 4. Diffusion of [Ru(Bpy)₃]²⁺ Through a Model Gelatin Hydrogel and Cadaver Corneal Stroma

The rate of diffusion of Ru(bpy)₃ through both fresh bovine cadaver corneal stroma and through a thermally gelled 12% gelatin hydrogel (as a model corneal stromal matrix), was studied. The results showed that a PBS solution containing 0.5 mM Ru(bpy)₃ allowed the free diffusion of Ru(bpy)₃ to a depth of 400 μm through an 800 μm mm thick bovine cadaver corneal stroma within 6 minutes. By comparison, the standard protocol for crosslinking corneal proteins, as conventionally used the Dresden CXL method, consists of 0.1% riboflavin solution (vitamin B2) applied to a de-epithelized cornea for 30 min to facilitate diffusion into the stroma to a depth of ~200 μm.

### Rate of Ru(Bpy)₃ Diffusion in Corneal Stroma

The observed rate of Ru(bpy)₃ diffusion through the stroma should require no more than 3 minutes of diffusion, followed by 1 sec of blue light illumination to cause photochemical crosslinking of intrinsic stromal proteins. The entire crosslinking procedure is therefore reduced from 30 minutes per eye (current treatment) to less than 3 minutes per eye (the treatment described in the present disclosure). This will allow the surgeon to carry out procedures at a 10-fold faster rate in the operating theatre, and patients will be subject to much briefer, less painful, surgical procedures.

Removing Bound [Ru(Bpy)₃]²⁺ from Corneal Stroma Following Photochemical Crosslinking.

Literature reports show that Ru(bpy)₃ does not bind to protein hydrogels (Kurimura et al., 1982). Nevertheless, interaction between Ru(bpy)₃ and corneal stromal proteins might result in residual non-covalent binding of the Ru(bpy)₃ within the crosslinked stromal hydrogel, causing temporary colouring of the stroma. This temporary colouring can be addressed by rinsing the crosslinked cornea with a solution (delivered as eye drops) containing low molecular weight (PSS)—poly(styrenesulfonic acid), sodium salt (e.g. [MW 4,600 Da] poly(p-styrenesulphonate) from: Polysciences Cat. No. 16248-250—https://www.polysciences.com/german/polystyrenesulfonic-acid-sodium-salt-mw-4600). Poly(p-styrenesulfonic acid) is a polystyrene polymer that is used as a cation exchange resin. The strong charge and hydrophobic interaction between the bipyridyl ligands of the Ru(bpy)₃ complex and PSS will effectively extract the residual Ru(bpy)₃ from the cured hydrogel thereby removing residual retained Ru(bpy)₃ (Tuite et al., 2012).

### Example 5. Diffusion of Ru(Bpy)₃ in 12% Gelatin Gel

A 12% Gelatin (Sigma G1890) Type A, 300 Bloom solution was prepared in PBS (pH7.4) by melting at 50° C. 60 μL was added to an 8.5 mm (diameter)×1 mm (deep) round Teflon mould. The Gelatin was allowed to set thermally for 10 min at 18.5° C. At 10 min, 60 μL 0.5 mM Ru(bpy)₃ (0.5 mM solution in PBS) is added to the top of the thermally gelled 12% gelatin disc. The gelatin disc was covered with foil during diffusion time. Excess solution was blotted from the top of the gel using a Kimwipe. Remove gelled disc using spatula, add to 940 μL of PBS @55° C. Melt gel (30 sec)—add solution to cuvette, read A₄₅₂.

If diffusion is expressed as a percentage of the total disc volume equilibrated with the applied 0.5 mM Ru(bpy)₃ solution, then, assuming total mixing of the applied 60 μL solution with the 60 μL gelatin disc, 100% diffusion would represent an A₄₅₂ of 0.205 [0.221−0.016=gelatin−PBS blank)]. So, diffusion to 30% of the hydrogel depth/thickness (in this case representing ca. 300 μm) would be attained in about 2.3 min. This is the desired depth of penetration of a crosslinking agent in the human cornea (by comparison with Riboflavin-based CXL methods, which require 30 minutes of diffusion).

### Example 6. Ru/SPS Diffusion Model: Diffusion Rate Through Thermally Set (10 Min @RT) 12% Gelatin/PBS Hydrogel

Aim: The aim of these experiments was to describe the rate of diffusion of solutions (i) or (ii) (below) through a thermally-gelled gelatin hydrogel, representing an in vitro model of the corneal stroma:
(i) Ru(bpy)₃ (0.5 mM, 1.0 mM, 2.0 mM—proposed concentrations); and
(ii) Riboflavin (2.6 mM) 0.1% (currently used concentration in the FDA-approved CXL crosslinking treatment).

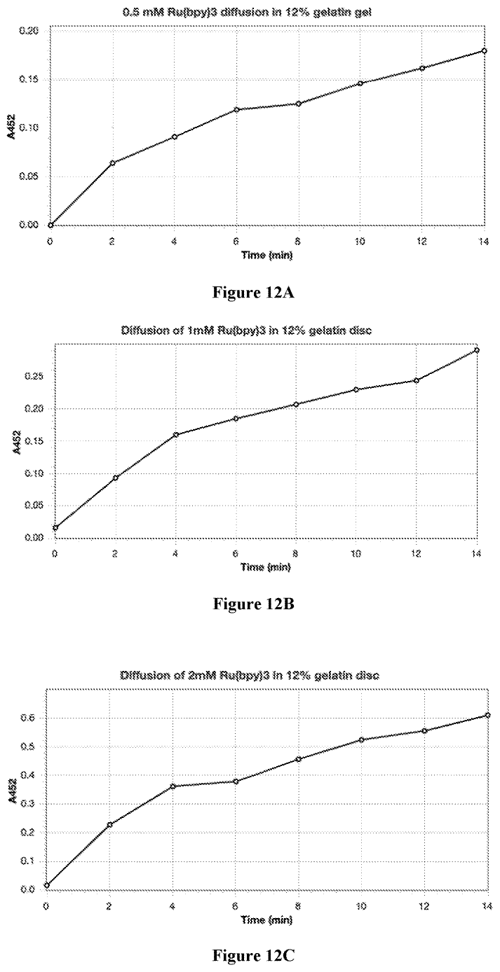

Method: 12% Gelatin (Sigma G1890) Type A 300 Bloom in PBS (pH 7.4), was melted at 50° C. and 60 μL added to an 8.5 mm (diameter) disc×1 mm (deep) Teflon mould. The gelatin was allowed to set thermally for 10 min at 18.5° C. 60 μL of Ru(bpy)₃/SPS solution (either a 0.5 mM Ru(bpy)₃+50 mM SPS; a 1.0 mM Ru(bpy)₃+50 mM SPS or a 2.0 mM Ru(bpy)₃+50 mM SPS) was added and allowed to diffuse into the gel disc for varying times (0, 2, 4, 6, 8, 10, 14 min). Excess liquid was blotted from the top surface of the mould. At the end point of the diffusion period, the thermally set gelatin hydrogel was illuminated using a 3M ESPE blue LED lamp (1,200 mW/cm²) for 5 sec at 1 cm distance from the light guide tip. The crosslinked gelatin hydrogel was weighed on a microbalance before being placed into a 65° C. water bath for 30 sec. Excess liquid was removed from the crosslinked hydrogel and it was again weighed to measure residual crosslinked gelatin hydrogel. A plot was prepared showing % crosslinked gelatin vs diffusion time (zero to 14 min). Diffusion of 0.5 mM, 1.0 mM and 2.0 mM Ru(bpy)₃ into gelatin discs is shown in FIGS. 12A to 12C.

### Example 7. Diffusion of Ru(Bpy)₃+SPS: Photochemical Crosslinking of Gelatin Hydrogel

Aim: To determine the time required for a solution containing Ru(bpy)₃+SPS to diffuse through a 1 mm thick sheet of thermally-gelled gelatin and then to form covalent crosslinks between gelatin molecules, thus rendering the hydrogel resistant to subsequent melting at high temperature. This is a test to demonstrate the time required for covalent photochemical crosslinking of gelatin molecules in the hydrogel.

Method: 60 μL of Ru(bpy)₃+SPS solution was allowed to diffuse into the thermally set (10 min at RT) 12% gelatin gel (60 μL in PBS buffer) for diffusion times of between 2 and 14 minutes. The gel was illuminated for 5 seconds with a 450 nm blue LED lamp. After crosslinking the gel was weighed and then immersed in a 65° C. water bath for 30 sec. Excess liquid was blotted from the gel and the gel was subsequently weighed.

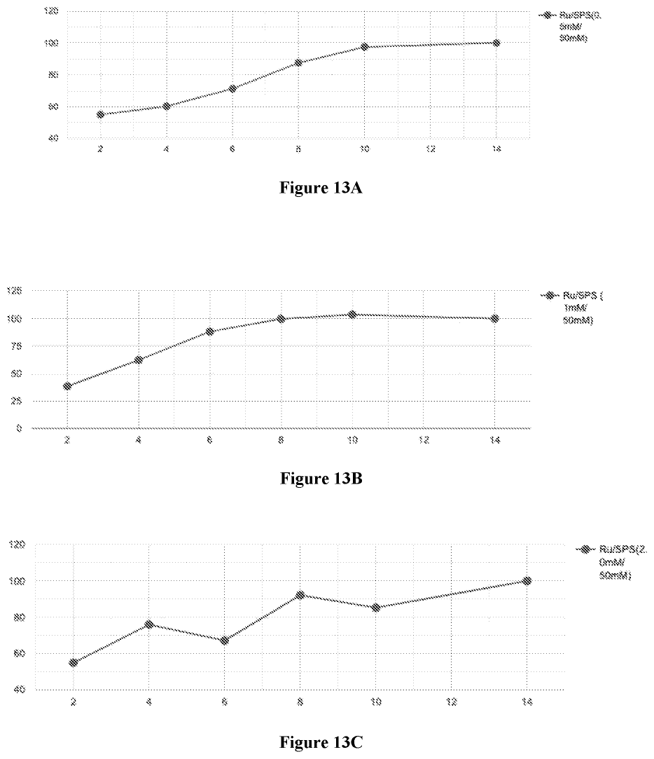

Any non-crosslinked gelatin chains will have dissolved in the hot water, and the covalently crosslinked chains will become part of a crosslinked polymeric gelatin hydrogel that is resistant to dissolution by heating. If all gelatin molecules are crosslinked, no gelatin will melt from the hydrogel and the weight of the gelatin hydrogel disc after heat treatment will equal the weight prior to heat treatment (this situation represents 100% crosslinking). Diffusion of 0.5 mM Ru(bpy)₃+50 mM SPS; 1.0 mM Ru(bpy)₃+50 mM SPS; and 2.0 mM Ru(bpy)₃+50 mM SPS into 12% gelatin discs (FIGS. 13A, 13B, and 13C, respectively).

Results: These investigations demonstrate that Ru(bpy)₃ can diffuse through the entire 1.0 mm thickness of a 12% (w/v) gelatin hydrogel within 6 minutes. Following the diffusion step, the entire gelatin hydrogel can be covalently crosslinked by a 5 second illumination with a 450 nm blue LED lamp. The thickness of the human cornea is about 450 μm, so a diffusion time of about 3 minutes followed by a 5 second illumination is sufficient to form a completely crosslinked corneal stroma.

Diffusion of Riboflavin into 12% Gelatin Gel

For comparison, the present inventors have also investigated the diffusion of Riboflavin (0.1%) through the 12% gelatin hydrogel model. The rate of Riboflavin diffusion is slightly slower than the rate of diffusion of a 0.5 mM Ru(bpy)₃ solution. The current Riboflavin/UV-A CXL method requires a 30-minute Riboflavin diffusion period followed by a 10 minute (accelerated) or 30-minute (traditional Dresden protocol) UV-A illumination period.

Riboflavin used at 0.1% for CXL
≡ 0.1 g/100 ml
≡ 1 g/L
≡ 1 mg/ml
=1/376.37 mol
=2.6 mM in PBS (pH 7.4) - not fully soluble in PBS*
*Added 1 μL of 10N NaOH to 2 ml of 0.1% solution in PBS - took pH to ca. 8.5 (by pH paper test). Riboflavin solution was now fully soluble at 0.1% (2.6 mM) at RT.

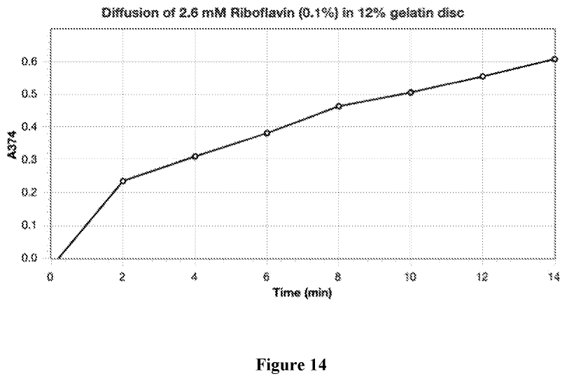

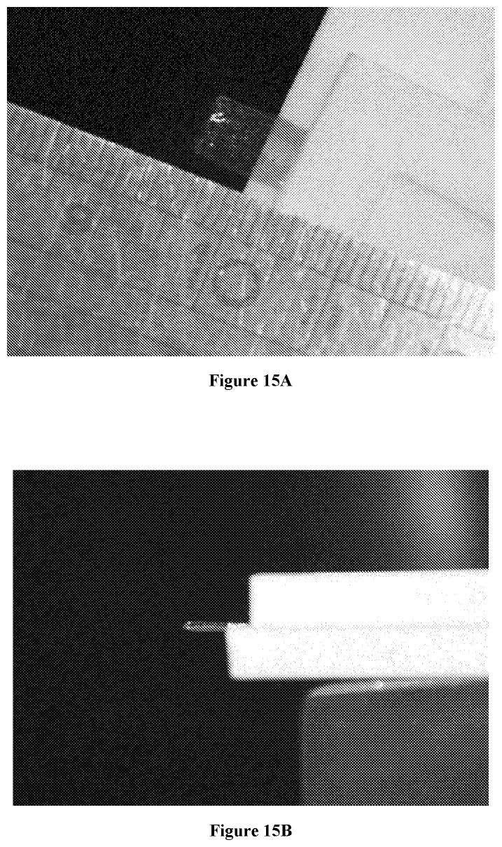

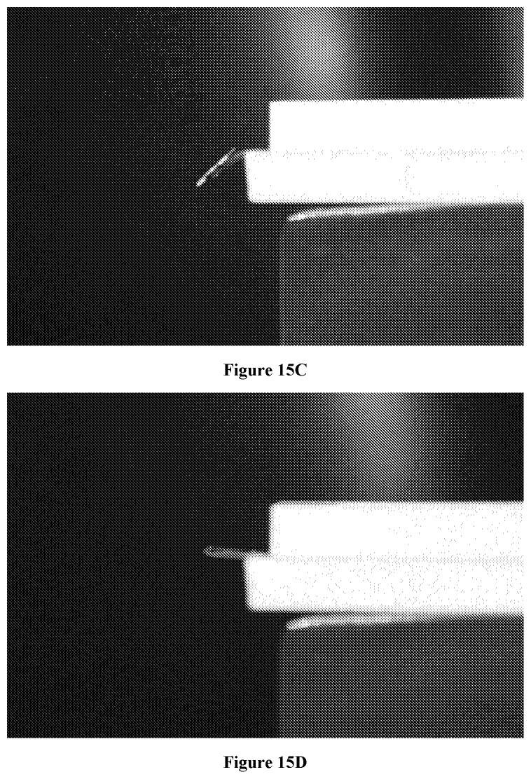

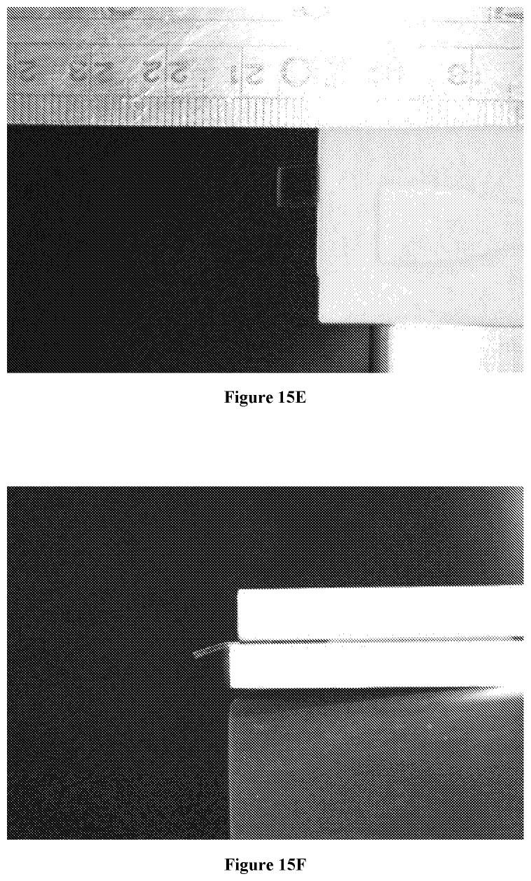

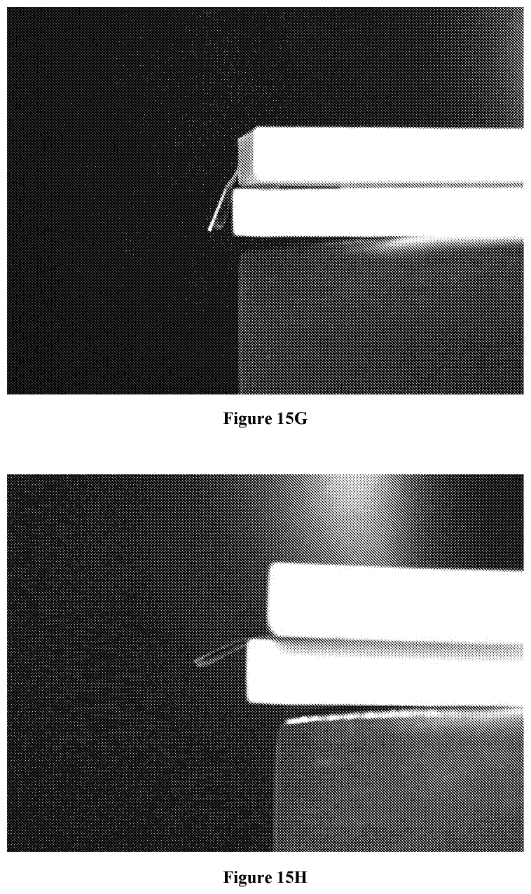

FIG. 14 shows the diffusion of 2.6 mM (0.1%) Riboflavin into 12% gelatin gel over time up to 14 minutes.

### Example 8. Gelatin “Molecular Linker”—Enhanced Mechanical Properties

The intrinsic crosslinking of a thermally-gelled 12% gelatin (Sigma G-1890—high molecular weight Type A gelatin) hydrogel was compared using either:
A. A solution of 1 mM Ru(bpy)₃+50 mM SPS alone
B. A solution containing autoclaved (size-selected) BH-derivatised Nippi MediGelatin (150 mg/ml)+1 mM Ru(bpy)₃+50 mM SPS.

### Materials and Methods

A highly tyrosine-derivatised LMW gelatin sample (BH Nippi MediGelatin sample containing 3.8 mol % tyrosine) was resuspended at 100 mg/ml in water and heated at 50° C. until dissolved. The solution was autoclaved for 7 successive cycles of 120 min each. This procedure was carried out in light of the study by Vanhoecke and Olijve (2018) which described the effect of autoclaving on the molecular weight of gelatin—a single cycle of autoclaving reduced the molecular weight of G1890 Sigma gelatin from 150 kDa to 75 kDa. On this basis, the inventors carried out 7 successive cycles of autoclaving of a G1890 gelatin solution (in 1×PBS) at 100 mg/ml. After 4 autoclave cycles, the G1890 gelatin sample (at 100 mg/ml) did not gel at 4° C. The study by Mrak and Stewart (1957) showed that “The molecular weight of a degraded gelatin which did not gel at 0° C. is around 10 kDa to 15 kDa”.

Following autoclaving, approximately 2 μL-5 μL samples (of the 100 mg/ml solution) were taken and frozen. SDS-PAGE was conducted to analyse the success of autoclaving in reducing the M_{w} of gelatin to approximately 10 kDa to 15 kDa. See SDS-PAGE image (FIG. 2) showing Mw range of autoclaved gelatin with respect to autoclave time. The aim is to generate samples of BH-derivatised gelatin that show an average Mw range of 10 kDa to 15 kDa.

### Example 9. Effect of Autoclaved Gelatin Molecular Linker on the Elastic Modulus of Crosslinked Gelatin Hydrogel

Methods. A 12% (w/v) solution of Sigma G1890 (Type A) gelatin was prepared by dissolving solid gelatin in PBS buffer at 50° C. The solution was maintained at 40° C. until used to cast thermally-set hydrogels. The molten solution was pipetted into a Teflon mould in order to prepare a 1 mm thick sheet of gelatin hydrogel of dimensions 10 mm×5 mm×1 mm (50 μL volume).

Two experiments were carried out to assess the effect of inclusion of the tyrosine-derivatised molecular linker on the mechanical properties of the crosslinked hydrogel. The thermally-set gelatin strip was soaked for 10 minutes in a solution (60 μL) containing either of the 2 following solutions (A) and (B). After a 10 minute diffusion period, excess liquid was blotted from the crosslinked gelatin strip using a Kimwipe tissue and the gelatin strip was photocrosslinked for a 5 sec irradiation period using a 450 nm blue LED dental lamp (3M ESPE blue LED lamp (1,200 mW/cm²) at 5 cm distance from the light guide tip).

(A). The 1 mm thick, 10 mm×5 mm 12% gelatin strip was crosslinked in the presence of 7× autoclaved BH-derivatised Nippi gelatin (150 mg/ml) with 1 mM Ru(bpy)₃+50 mM SPS in the photochemical crosslinking reaction (total volume=60 μL), or

(B). The 1 mm thick, 10 mm×5 mm 12% gelatin strip was crosslinked in the presence of 1 mM Ru(bpy)₃+50 mM SPS only included in the photochemical crosslinking reaction (total volume=60 μL). No tyrosine-derivatised molecular linker solution was added during the crosslinking reaction.

The photochemically-crosslinked gelatin strip was arranged so that a 5 mm length of the strip was cantilevered over an edge of a PTFE block. A 150 mg weight was added to the protruding strip. A series of photographs were taken to determine the weight-bearing capacity of the cantilevered gelatin strips.

A. The 1 mm thick, 10 mm×5 mm 12% gelatin strip was crosslinked in the presence of 7× autoclaved BH-derivatised Nippi gelatin (150 mg/ml) with 1 mM Ru(bpy)3+50 mM SPS in the photochemical crosslinking reaction (total volume=60 μL).

B. The 1 mm thick, 10 mm×5 mm 12% gelatin strip was crosslinked in the presence of 1 mM Ru(bpy)₃+50 mM SPS ONLY included in the photochemical crosslinking reaction (total volume=60 μL). No tyrosine-derivatised molecular linker solution was added during the crosslinking reaction.

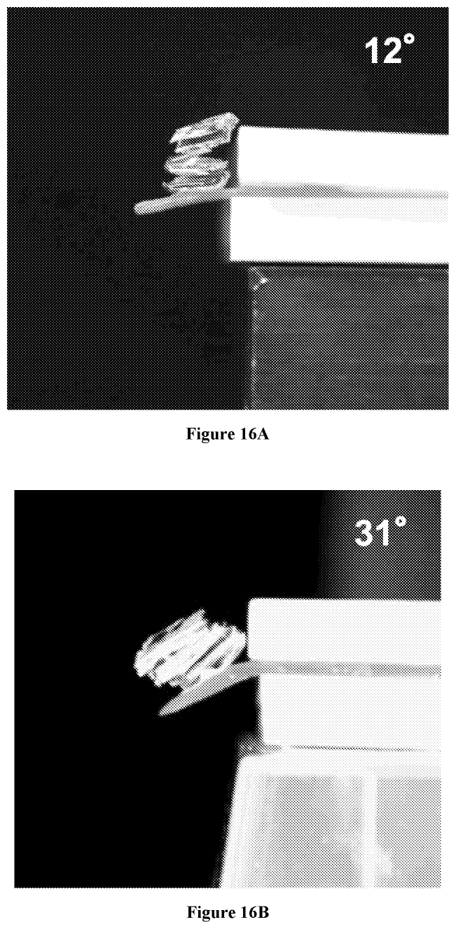

Results. The present inventors demonstrated that a thin (1 mm) gelatin hydrogel sheet photochemically crosslinked in the presence of Low Molecular Weight (LMW—degraded gelatin of MW ca. 10 kDa-15 kDa) tyrosine-derivatised gelatin molecules, is able to support a greater mass with less displacement compared to an identical gelatin gel crosslinked with only Ru(bpy)₃+SPS (FIGS. 16A-16H).

These results demonstrate that addition of a solution (150 mg/ml) of degraded (tyrosine-derivatised) gelatin to the Ru(bpy)₃+SPS photochemical reagent solution, improves the mechanical properties of the crosslinked gelatin hydrogel, compared to the mechanical performance of a gelatin hydrogel photochemically crosslinked with a solution of Ru(bpy)₃+SPS alone. The gelatin hydrogel model displays a higher elastic modulus when photochemically crosslinked in the presence of the LMW tyrosine-derivatised gelatin molecular linker.

### Example 10. Effect of autoclaved BH-derivatised gelatin molecular linker on the elastic modulus of bovine cornea

Methods Bovine corneal “buttons” were prepared by dissecting the corneas from fresh (collected on ice and prepared within 4 hours of slaughter) abattoir specimens. The corneal epithelium was removed from the entire surface of the cornea prior to dissection of the cornea. The freshly prepared corneas were rinsed in saline and blotted dry using a Kimwipe tissue.

Two experiments were carried out to assess the effect of inclusion of the autoclaved, low molecular weight tyrosine-derivatised molecular linker on the mechanical properties of the crosslinked hydrogel. The bovine cornea was soaked for 10 minutes in a solution (60 μL), containing either of the 2 following solutions (A) and (B). After a 10-minute diffusion period, excess liquid was blotted from the crosslinked gelatin strip using a Kimwipe tissue and the gelatin strip was photocrosslinked for a 5 sec irradiation period using a 450 nm blue LED dental lamp (The Elipar™ S10 LED Curing Light (1,200 mW/cm²) at 5 cm distance from the light guide tip).

The photochemically-crosslinked corneal strips were arranged so that a 10 mm length of the strip was cantilevered over an edge of a PTFE block. A 111 mg weight was added to the protruding strip. A series of photographs were taken to determine the weight-bearing capacity of the cantilevered bovine corneal strips.

A. (FIG. 16A) The bovine cornea (10 mm×5 mm) strip was crosslinked in the presence of 7× autoclaved BH-derivatised Nippi gelatin (150 mg/ml) with 1 mM Ru(bpy)₃+50 mM SPS in the photochemical crosslinking reaction (total volume=60 μL).

B. (FIG. 16 B) The bovine cornea (10 mm×5 mm) strip was crosslinked in the presence of 1 mM Ru(bpy)3+50 mM SPS ONLY included in the photochemical crosslinking reaction (total volume=60 μL). No tyrosine-derivatised molecular linker solution was added during the crosslinking reaction.

### Results

The present inventors demonstrated that a bovine cornea strip (5 mm×10 mm) photochemically crosslinked in the presence of Low Molecular Weight (LMW—degraded gelatin of MW ca. 12 kDa-20 kDa) tyrosine-derivatised gelatin molecules, is able to support an equivalent mass with less vertical displacement (FIGS. 16 A) compared to an identical corneal strip crosslinked with ONLY Ru(bpy)₃+SPS (FIG. 16 B). Note that the angle of deflection of the cantilevered strip crosslinked in presence of the LMW BH-derivatised gelatin “Molecular Linker” is lower (FIG. 16 A—12°) than the deflection recorded for the strip crosslinked in the absence of the LMW-BH-derivatised gelatin molecular linker (FIG. 16 B—31°).

These results demonstrate that addition of a solution (150 mg/ml) of degraded (tyrosine-derivatised) gelatin (Molecular weight 12 kDa-20 kDa) to the Ru(bpy)₃+SPS photochemical reagent solution, improves the mechanical properties of the crosslinked gelatin hydrogel, compared to the mechanical performance of a bovine cornea photochemically crosslinked with a solution of Ru(bpy)₃+SPS alone. The photocrosslinked corneal model displays a higher elastic modulus when photochemically crosslinked in the presence of the LMW tyrosine-derivatised gelatin molecular linker (“molecular staple”), compared to the corneal strip photochemically crosslinked in the absence of the low molecular weight tyrosine derivatised gelatin molecular linker.

The present inventors have shown that the elastic modulus of the bovine cornea crosslinked in the presence of degraded, (BH-derivatised) LMW gelatin (Molecular weight 12 kDa-20 kDa) molecular linker (a solution of 150 mg/ml) was 5.3-fold higher than the bovine cornea crosslinked in the absence of the molecular linker.

The calculation of elastic modulus (E) for both experiments were:

$$E ( crosslinked with no molecular linker ) \text{∼=} 266.23 kPa$$

$$E\_ ( crosslinked with molecular linker ) \text{∼=} 1.41 MPa$$

Calculation of the elastic modulus (E) from “cantilevered beams” experiments is possible using the Modulus calculation notes below:

$$Beam Dimensions = 5 mm ( w ) \times 0.8 mm ( h ) \times 10 mm ( l )$$

$$Assume that the density of the beam material is \text{∼=} 1.05 g / {cm}^{⋀} 3$$

Modelling the mass of the beam and the aluminium foil mass as a point mass in the center of the beam we get 153 mg at 5 mm from the pivot of the beam.
Convert this mass to Newtons (=0.0014994) and mm to m to get moment in Nm units: Total moment at equilibrium of the beam at the pivot is 0.0014994N*0.005 m=0.000007497 Nm.
Second moment of area is a multiple integral over the squared distance from the bending axis of the beam. Since the beam is approximately a rectangular cross-section beam, the formula bh\hat{ }3/12 (which accounts for the squared distance both below and above the bending axis) is appropriate. Hence the second moment of area for this beam is (0.0008\hat{ }3)0.005/12=2.13e10\hat{ }-13m\hat{ }4.

$$B\_untreated := beam before linker treatment$$

$$B\_treated := beam after linker treatment$$

Curvature of B_untreated at the pivot obtained by overlaying osculating circle and finding its radius. Such a curvature is found to be 0.132(mm/mm)/mm=0.132 mm\hat{ }-1=132 m\hat{ }-1
Similarly the curvature of B_treated at the pivot is found to be 0.0249(mm/mm)/mm=0.0249 mm\hat{ }-1=24.9 m\hat{ }-1

$$E\_untreated := modulus before linker treatment$$

$$E\_treated := modulus after linker treatment$$

Using the equation M=EIk (M is moment at pivot, E is flexural modulus, I is second moment of area, k is curvature),

### Therefore,

$$E\_ ( no molecular linker ) \text{\textasciitilde =} 266.23 kPa$$

$$E\_ ( + molecular linker ) \text{\textasciitilde =} 1.41 MPa$$

A comparison of the photochemical crosslinking method presented by the current inventors (using blue LED light at 455 nm in the presence of Ru(bpy)3+SPS) to the traditional CXL method can be made by reference to the study published by Alenezi et al., (2022). These investigators measured the elastic modulus (E) of various segments of the human cornea using a nano-indentation method. They reported the elastic modulus (E) of the CXL-crosslinked anterior segment of the human cornea was 178% higher than the elastic modulus of uncrosslinked anterior segment of the cornea. The actual elastic modulus (E) reported for the Riboflavin-UV-A based CXL-treated anterior segment of younger cornea was 285 kPa. In other words, the photochemical crosslinking method, carried out using only Ru(bpy)3+SPS alone, yielded (in 3 minutes, including a 5 second irradiation with 455 nm blue LED light) an elastic modulus almost equal to that achieved after a total of 39 minutes of treatment per cornea (30 min riboflavin application, followed by 9 minutes of UV-A (10 mW/cm2) for 9 min, for total irradiation of 5.4 J/cm2). Significantly, addition of a solution containing 150 mg/ml of degraded (12 kDa-20 kDa) BH-derivatised gelatin, resulted in a 5.3-fold increase in elastic modulus (E) to 1.41 MPa.

In order to achieve the desired increase in elastic modulus following Ru(bpy)₃+SPS mediated crosslinking method, a person skilled in the art, would be able to tune the extent of photochemical crosslinking desired (and thus the level of crosslinking causing an increase in the elastic modulus) by varying the amount of each of the components of the photochemical crosslinking method. For example, the amount of crosslinking induced by the photochemical crosslinking method described here can be changed by varying (i) the Ru(bpy)₃ concentration from about 0.25 mM to about 2 mM, (ii) varying the SPS concentration from about 10 mM to about 100 mM and (iii) varying the concentration of the BH-derivatised molecular linker from about 10 mg/ml to about 200 mg/ml. Additionally, the illumination time can be varied from about 0.5 seconds to about 10 seconds, which has the effect of changing the blue light energy transmitted to the cornea. This can also be achieved by varying the distance of the light source from the corneal surface from about 20 mm to about 200 mm.

It was concluded that the short tyrosine-derivatised gelatin chains can form crosslinks between distantly separated gelatin chains (as described in Experiment 9) within the thermally gelled hydrogel, or between distantly separated collagen chains in bovine cornea (as described in Experiment 10). Such crosslinks contribute to the enhanced mechanical performance when the gelatin molecular linker is present during the photochemical crosslinking reaction. In both experiments 9 and 10, inclusion of the low molecular weight tyrosine derivatised gelatin molecular linker, during the diffusion of crosslinking reagents (Ru(bpy)₃+SPS), resulted in an increase in the apparent elastic modulus of either the thermally gelled gelatin strip (Experiment 9) or in the freshly prepared corneal tissue strip (Experiment 10). In the absence of the molecular linker, dityrosine crosslinks can only form between closely associated tyrosine residues, located on closely (<5 Å) adjacent gelatin chains (in Experiment 9) or between tyrosine residues located on closely associated proteoglycan and collagen chains (in Experiment 10), through short-range hydrophobic 7r interactions.

In both Experiments 9 and 10, the inclusion of the tyrosine-derivatised low molecular weight molecular linker was shown to increase the elastic modulus of the test material; either, thermally gelled gelatin strip (in Experiment 9), or, bovine cornea (in Experiment 10). This increase in material stiffness resulted from the introduction of new intermolecular crosslinks induced by the presence of the tyrosine-derivatised low molecular weight molecular linker.

The present application claims priority from Australian provisional application no. 2023900571.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

All publications discussed and/or referenced herein are incorporated herein in their entirety.

### REFERENCES

Akhtar et al., (2013), Acta Ophthalmol 86: 764-772
Alenezi et al., (2022) Experimental Eye Research 214: 108839
Bolton and Hunter, (1973), Biochem J. 133: 529-539
Brummer et al., (2011), Invest Ophthalmol Vis Sci. 52: 6363-6369
Buffiere et al., (2016) Green Chem. 18: 6516-6525
Cui et al., (2011), Invest Ophthalmol Vis Sci. 52: 2556-2564
Dervenis et al., (2020), Med Princ Pract 29: 332-337
Elvin et al., (2010), Biomaterials 31: 8323-8331
Escalante et al. (2012) Carbohydrate Polymers 87: 42381-2387
Forrester et al., (2020) The Eye: basic sciences in practice (5th Edition Elsevier)
Habibi et al., (2010) Chemical Reviews, 110(6), 3479-3500
Hayes et al., (2013), PLoS ONE 8: e52860
Heinze et al., (2016) Macromol Biosci 16: 10-42
Jardine (2022) Current Research in Green and Sustainable Chemistry 5: 100309
Kamaev et al. (2012) Invest Ophthalmol. Vis. Sci. 53: 2360-2367
Khaire et al., (2022) Industrial Crops and Products 187: 115545
Kurimura et al., (1982), Bull Chem Soc Jpn 55: 55-58
Kuzmenko et al., (2014) Carbohydr Polym. 102: 862-868
Lima et al., (2004) Macromolecular Rapid Communications, 25, 771-787
Markstedt et al., (2017) Carbohydr Polym. 157: 1349-1357)
Miller, (1996), Biochem Biophys Res Commun. 218: 377-382
Mrak and Stewart (1957), Advances in Food Research (Academic Press) p 307
O'Brart et al., (2015), Am J Ophthalmol. 160: 1154-1163
Prausnitz and Noonan, (1998), J Pharm Sci 87: 1479.
Raiskup-Wolf et al., (2008), J Cataract Refract Surg. 34: 796-801
Rajapaksha et al., (2015), Molecular and Cellular Biomechanics. 12: 215-230
Seiler et al., (2014), Invest Ophthalmol Vis Sci. 56: 6740-6746
Shaghaleh et al., (2021) Journal of Cleaner Production 297: 126664
Sharma et al., (2012), Am J Ophthalmol. 154: 922-926
Simon et al., (2023) Biomacromolecules 24: 166-177
Sliney, D. et al., (2005) Applied Optics 44: 2162-2176.
Spoerl et al., (1998), Ex Eye Res. 66: 97-103
Spoerl et al., (2004a), Curr Eye Res. 29: 35-40
Spoerl et al., (2004b), Ophthalmo-logica 218: 136-140
Tuite et al., (2012), Phys. Chem. Chem. Phys. 14: 3681-3692
van Dijk et al., (2018), Graefes Arch. Clin. Exp. Ophthalmol. 256: 1151-1158
Vanhoecke and Olijve (2018), Rousselot Report, 2018
Vimalin et al., (2012), Cornea 31: 1052-1059
Wollensak et al., (2003), Ophthalmologe. 100: 44-49
Wollensak et al., (2004), Cornea. 23: 503-507
Xu et al., (2014), International Journal of Biological Macromolecules 67: 205-209
Ziółkowska, et al., (2023) Cells 12: 1014

1. A method of crosslinking endogenous protein in a tissue, the method comprising:
(i) contacting the tissue with a molecular linker, a photoactivatable metal ligand complex, and an electron acceptor, whereby the molecular linker contacts the endogenous protein in the tissue; and
(ii) irradiating the tissue.

2. The method of claim 1, wherein the molecular linker is a polymer comprising two or more tyrosine and/or tyramine residues.

3. The method of claim 2, wherein the polymer comprising one or more tyrosine and/or tyramine residues is selected from tyrosine and/or tyramine-derivatised gelatin, tyrosine and/or tyramine-derivatised dextran, and/or tyrosine and/or tyramine-derivatised xylan and/or tyrosine and/or tyramine-derivatised low molecular weight nanocellulose.

4. The method of any one of claims 1-3, wherein the molecular linker has a molecular weight between about 5 kDa to about 50 kDa.

5. The method of any one of claims 1-4, wherein the photoactivatable metal ligand complex is ruthenium (tris)bipyridyl chloride.

6. The method of any one of claims 1-5, wherein the electron acceptor is sodium persulphate.

7. The method of any one of claims 1-6, wherein the tissue is corneal tissue.

8. The method of claim 7, wherein the step of contacting the corneal tissue with a molecular linker, a photoactivatable metal ligand complex, and an electron acceptor comprises administering a solution comprising the molecular linker, photoactivatable metal ligand complex, and electron acceptor to the surface of the cornea, whereby the solution diffuses into the corneal stroma.

9. The method of any one of claims 1-8, wherein the tissue is irradiated with light having a wavelength of between around 430 nm to 480 nm.

10. The method of any one of claims 1-9, wherein irradiating the tissue comprises delivering a light intensity of between about 200 mW/cm² and 1,200 mW/cm² to the tissue surface.

11. The method of claim of any one of claims 9-10, wherein the tissue is irradiated for about 1 to about 10 seconds.

12. A method of treating a weakened corneal stroma in a subject, the method comprising:
(i) administering a molecular linker, a photoactivatable metal ligand complex, and an electron acceptor to a cornea of the subject; and
(ii) irradiating the cornea.

13. A method of treating or preventing at least one symptom of keratoconus in a subject, the method comprising:
(i) administering a molecular linker, a photoactivatable metal ligand complex, and an electron acceptor to a cornea of the subject; and
(ii) irradiating the cornea.

14. The method of claim 12 or claim 13, wherein the molecular linker is a polymer comprising two or more tyrosine and/or tyramine residues.

15. The method of claim 14, wherein the polymer comprising two or more tyrosine and/or tyramine residues is selected from tyrosine and/or tyramine-derivatised gelatin, tyrosine and/or tyramine-derivatised dextran, and/or tyrosine and/or tyramine-derivatised xylan and/or tyrosine and/or tyramine-derivatised low molecular weight nanocellulose.

16. The method of any one of claims 12-15, wherein the molecular linker has a molecular weight between about 5 kDa to about 50 kDa.

17. The method of any one of claims 12-16, wherein the photoactivatable metal ligand complex is ruthenium (tris)bipyridyl chloride.

18. The method of any one of claims 12-17, wherein the electron acceptor is sodium persulphate.

19. The method of any one of any one of claims 12-18, wherein the step of administering the molecular linker, photoactivatable metal ligand complex, and electron acceptor comprises administering a solution comprising the molecular linker, photoactivatable metal ligand complex, and electron acceptor to the surface of the cornea, whereby the solution diffuses into the corneal stroma.

20. The method of claim 19, wherein the solution diffuses into the corneal stroma for up to about 5 minutes.

21. The method of any one of claims 12-20, wherein the cornea is irradiated with light having a wavelength of between around 430 nm to 480 nm.

22. The method of claim of claim 21, wherein the tissue is irradiated for about 1 to about 10 seconds.

23. The method of any one of claims 12-22, wherein cross-linking the molecular linker and endogenous protein results in an improvement in one or more mechanical properties of the corneal stroma.

24. Use of a molecular linker in the manufacture of a medicament for the treatment or prevention of one or more symptoms of disease in a subject.

25. A composition for the treatment of keratoconus in a subject, the composition comprising a molecular linker, a photoactivatable metal ligand complex, and an electron acceptor.

26. A composition for intermolecular crosslinking between endogenous stromal collagen fibrils and other endogenous proteins in the cornea, wherein the composition comprises a molecular linker, a photoactivatable metal ligand complex, and an electron acceptor.


SEQUENCE LISTING (13 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1464 aa, synthetic construct
MFSFVDLRLLLLLAATALLTHGQEEGQVEGQDEDIPPITCVQNGLRYHDRDVWYPEPCRICVCDNGYVLCDDVICDETYNCPGAEVPEGECCPVCPDGSESPTDQETTGVEGPYGDTGPRGPRGPAGPPGRDGIPGQPGLPGPPGPPGPPGPPGLGGNFAPQLSYGYDEYSTGGISVPGPMGPSGPRGLPGPPGAPGPQGFQGPPGEPGEPGASGPMGPRGPPGPPGYNGDDGEAGYPGRPGERGPPGPQGARGLPGTAGLPGMYGHRGFSGLDGAYGDAGPAGPYGEPGSPGENGAPGQMGPRGLPGERGRPGAPGPAGARGNDGATGAAGPPGPTGPAGPPGFPGAVGAYGEAGPQGPRGSEGPQGVRGEPGPPGPAGAAGPAGNPGADGQPGAYGANGAPGIAGAPGFPGARGPSGPQGPGGPPGPYGNSGEPGAPGSYGDTGAYGEPGPVGVQGPPGPAGEEGYRGARGEPGPTGLPGPPGERGGPGSRGFPGADGVAGPYGPAGERGSPGPAGPYGSPGEAGRPGEAGLPGAYGLTGSPGSPGPDGYTGPPGPAGQDGRPGPPGPPGARGQAGVMGFPGPYGAAGEPGYAGERGVPGPPGAVGPAGYDGEAGAQGPPGPAGPAGERGEQGPAGSPGFQGLPGPAGPPGEAGYPGEQGVPGDLGAPGPSGARGERGFPGERGVQGPPGPAGPRGANGAPGNDGAYGDAGAPGAPGSQGAPGLQGMPGERGAAGLPGPYGDRGDAGPYGADGSPGYDGVRGLTGPIGPPGPAGAPGDYGESGPSGPAGPTGARGAPGDRGEPGPPGPAGFAGPPGADGQPGAYGEPGDAGAYGDAGPPGPAGPAGPPGPIGNVGAPGAYGARGSAGPPGATGFPGAAGRVGPPGPSGNAGPPGPPGPAGYEGGYGPRGETGPAGRPGEVGPPGPPGPAGEYGSPGADGPAGAPGTPGPQGIAGQRGVVGLPGQRGERGFPGLPGPSGEPGYQGPSGASGERGPPGPMGPPGLAGPPGESGREGAPGAEGSPGRDGSPGAYGDRGETGPAGPPGAPGAPGAPGPVGPAGYSGDRGETGPAGPAGPVGPVGARGPAGPQGPRGDYGETGEQGDRGIYGHRGFSGLQGPPGPPGSPGEQGPSGASGPAGPRGPPGSAGAPGYDGLNGLPGPIGPPGPRGRTGDAGPVGPPGPPGPPGPPGPPSAGFDFSFLPQPPQEYAHDGGRYYRADDANVVRDRDLEVDTTLYSLSQQIENIRSPEGSRYNPARTCRDLYMCHSDWYSGEYWIDPNQGCNLDAIYVFCNMETGETCVYPTQPSVAQYNWYISYNPYDYRHVWFGESMTDGFQFEYGGQGSDPADVAIQLTFLRLMSTEASQNITYHCYNSVAYMDQQTGNLYYALLLQGSNEIEIRAEGNSRFTYSVTVDGCTSHTGAWGYTVIEYYTTYTSRLPIIDVAPLDVGAPDQEFGFDVGPVCFL

SEQ ID NO: 3 - protein, 1745 aa, Homo sapiens
MGNRRDLGQPRAGLCLLLAALQLLPGTQADPVDVLKALGVQGGQAGVPEGPGFCPQRTPEGDRAFRIGQASTLGIPTWELFPEGHFPENFSLLITLRGQPANQSVLLSIYDERGARQLGLALGPALGLLGDPFRPLPQQVNLTDGRWHRVAVSIDGEMVTLVADCEAQPPVLGHGPRFISIAGLTVLGTQDLGEKTFEGDIQELLISPDPQAAFQACERYLPDCDNLAPAATVAPQGEPETPRPRRKGKGKGRKKGRGRKGKGRKKNKEIWTSSPPPDSAENQTSTDIPKTETPAPNLPPTPTPLVVTSTVTTGLNATILERSLDPDSGTELGTLETKAAREDEEGDDSTMGPDFRAAEYPSRTQFQIFPGAGEKGAKGEPAVIEKGQQFEGPPGAPGPQGVVGPSGPPGPPGFPGDPGPPGPAGLPGIPGIDGIRGPPGTVIMMPFQFAGGSFKGPPVSFQQAQAQAVLQQTQLSMKGPPGPVGLTGRPGPVGLPGHPGLKGEEGAEGPQGPRGLQGPHGPPGRVGKMGRPGADGARGLPGDTGPKGDRGFDGLPGLPGEKGQRGDFGHVGQPGPPGEDGERGAEGPPGPTGQAGEPGPRGLLGPRGSPGPTGRPGVTGIDGAPGAKGNVGPPGEPGPPGQQGNHGSQGLPGPQGLIGTPGEKGPPGNPGIPGLPGSDGPLGHPGHEGPTGEKGAQGPPGSAGPPGYPGPRGVKGTSGNRGLQGEKGEKGEDGFPGFKGDVGLKGDQGKPGAPGPRGEDGPEGPKGQAGQAGEEGPPGSAGEKGKLGVPGLPGYPGRPGPKGSIGFPGPLGPIGEKGKSGKTGQPGLEGERGPPGSRGERGQPGATGQPGPKGDVGQDGAPGIPGEKGLPGLQGPPGFPGPKGPPGHQGKDGRPGHPGQRGELGFQGQTGPPGPAGVLGPQGKTGEVGPLGERGPPGPPGPPGEQGLPGLEGREGAKGELGPPGPLGKEGPAGLRGFPGPKGGPGDPGPTGLKGDKGPPGPVGANGSPGERGPLGPAGGIGLPGQSGSEGPVGPAGKKGSRGERGPPGPTGKDGIPGPLGPLGPPGAAGPSGEEGDKGDVGAPGHKGSKGDKGDAGPPGQPGIRGPAGHPGPPGADGAQGRRGPPGLFGQKGDDGVRGFVGVIGPPGLQGLPGPPGEKGEVGDVGSMGPHGAPGPRGPQGPTGSEGTPGLPGGVGQPGAVGEKGERGDAGDPGPPGAPGIPGPKGDIGEKGDSGPSGAAGPPGKKGPPGEDGAKGSVGPTGLPGDLGPPGDPGVSGIDGSPGEKGDPGDVGGPGPPGASGEPGAPGPPGKRGPSGHMGREGREGEKGAKGEPGPDGPPGRTGPMGARGPPGRVGPEGLRGIPGPVGEPGLLGAPGQMGPPGPLGPSGLPGLKGDTGPKGEKGHIGLIGLIGPPGEAGEKGDQGLPGVQGPPGPKGDPGPPGPIGSLGHPGPPGVAGPLGQKGSKGSPGSMGPRGDTGPAGPPGPPGAPAELHGLRRRRRFVPVPLPVVEGGLEEVLASLTSLSLELEQLRRPPGTAERPGLVCHELHRNHPHLPDGEYWIDPNQGCARDSFRVFCNFTAGGETCLYPDKKFEIVKLASWSKEKPGGWYSTFRRGKKFSYVDADGSPVNVVQLNFLKLLSATARQNFTYSCQNAAAWLDEATGDYSHSARFLGTNGEELSFNQTTAATVSVPQDGCRLRKGQTKTLFEFSSSRAGFLPLWDVAATDFGQTNQKFGFELGPVCFSS

SEQ ID NO: 4 - protein, 465 aa, Homo sapiens
STRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 5 - protein, 2446 aa, Homo sapiens
QAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 6 - protein, 3058 aa, Homo sapiens
RQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 7 - protein, 786 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGAPGAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 8 - protein, 2530 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACLRTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATLATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAATEESTEGPSATEVPSASEEPSPSEVPFPSEEPSPSEEPFPSVRPFPSVELFPSEEPFPSKEPSPSEEPSASEEPYTPSPPVPSWTELPSSGEESGAPDVSGDFTGSGDVSGHLDFSGQLSGDRASGLPSGDLDSSGLTSTVGSGLPVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGLPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGISGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAAAPARSCAEEPCGAGTCKETEGHVICLCPPGYTGEHCNIDQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTVACGEPPVVEHARTFGQKKDRYEINSLVRYQCTEGFVQRHMPTIRCQPSGHWEEPQITCTDPTTYKRRLQKRSSRHPRRSRPSTAH

SEQ ID NO: 9 - protein, 352 aa, Homo sapiens
MAGTICFIMWVLFITDTVWSRSVRQVYEVHDSDDWTIHDFECPMECFCPPSFPTALYCENRGLKEIPAIPSRIWYLYLQNNLIETIPEKPFENATQLRWINLNKNKITNYGIEKGALSQLKKLLFLFLEDNELEEVPSPLPRSLEQLQLARNKVSRIPQGTFSNLENLTLLDLQNNKLVDNAFQRDTFKGLKNLMQLNMAKNALRNMPPRLPANTMQLFLDNNSIEGIPENYFNVIPKVAFLRLNHNKLSDEGLPSRGFDVSSILDLQLSHNQLTKVPRISAHLQHLHLDHNKIKSVNVSVICPSPSMLPAERDSFSYGPHLRYLRLDGNEIKPPIPMALMTCFRLLQAVII

SEQ ID NO: 10 - protein, 338 aa, Homo sapiens
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN

SEQ ID NO: 11 - protein, 298 aa, Homo sapiens
MKTLQSTLLLLLLVPLIKPAPPTQQDSRIIYDYGTDNFEESIFSQDYEDKYLDGKNIKEKETVIIPNEKSLQLQKDEAITPLPPKKENDEMPTCLLCVCLSGSVYCEEVDIDAVPPLPKESAYLYARFNKIKKLTAKDFADIPNLRRLDFTGNLIEDIEDGTFSKLSLLEELSLAENQLLKLPVLPPKLTLFNAKYNKIKSRGIKANAFKKLNNLTFLYLDHNALESVPLNLPESLRVIHLQFNNIASITDDTFCKANDTSYIRDRIEEIRLEGNPIVLGKHPNSFICLKRLPIGSYF

SEQ ID NO: 12 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 13 - protein, 368 aa, Homo sapiens
MWPLWRLVSLLALSQALPFEQRGFWDFTLDDGPFMMNDEEASGADTSGVLDPDSVTPTYSAMCPFGCHCHLRVVQCSDLGLKSVPKEISPDTTLLDLQNNDISELRKDDFKGLQHLYALVLVNNKISKIHEKAFSPLRKLQKLYISKNHLVEIPPNLPSSLVELRIHDNRIRKVPKGVFSGLRNMNCIEMGGNPLENSGFEPGAFDGLKLNYLRISEAKLTGIPKDLPETLNELHLDHNKIQAIELEDLLRYSKLYRLGLGHNQIRMIENGSLSFLPTLRELHLDNNKLARVPSGLPDLKLLQVVYLHSNNITKVGVNDFCPMGFGVKRAYYNGISLFNNPVPYWEVQPATFRCVTDRLAIQFGNYKK